(19) United States
(12) Patent Application Publication
(10) Pub. No.: US 2022/0146518 A1
(43) Pub. Date: May 12, 2022

(54) TAG FOR LABELING BIOMOLECULES

(71) Applicant: Shanghai Polaris Biology Co., Ltd., Shanghai (CN)

(72) Inventors: Wenbin Wang, Shanghai (CN); Shuangwu Sun, Shanghai (CN); Ran Wei, Shanghai (CN); Yuchong Wang, Shanghai (CN)

(21) Appl. No.: 17/523,628

(22) Filed: Nov. 10, 2021

Related U.S. Application Data
(63) Continuation of application No. PCT/CN2020/089993, filed on May 13, 2020.

(30) Foreign Application Priority Data
May 13, 2019 (CN) PCT/CN2019/086623

(51) Int. Cl. G01N 33/58; G01N 33/68; C07K 16/30

(52) U.S. Cl. CPC G01N 33/58; G01N 33/6848; C07K 2317/52; C07K 2317/40; G01N 2560/00; C07K 16/30

(57) ABSTRACT

Provided is a tag for labeling a biomolecule, comprising a polymer backbone, one or more pendant moieties, and an end group capable of binding to the biomolecule, wherein each of the pendant moieties is attached to the polymer backbone and capable of chelating with an element. Also provided is a conjugate, comprising a biomolecule covalently coupled with the tag.

### CROSS-REFERENCE

This application is a continuation application of International Application No. PCT/CN2020/089993 filed on May 13, 2020, which claims priority from PCT Application No. PCT/CN2019/086623 which was filed on May 13, 2019, the content of which is hereby incorporated by reference in its entirety.

### BACKGROUND OF THE DISCLOSURE

Quantification of biomolecules is a great challenge but also offers promising opportunities for biological and biomedical applications including clinical and diagnostic testing. MS (Mass Spectrometry), especially ICP-MS (Inductively Coupled Plasma Mass Spectrometry), allows ultrasensitive quantification of elements, which offers an innovative approach for analysis of element tagged biomolecules.

### SUMMARY OF THE DISCLOSURE

In one aspect, the disclosure relates to a tag for labeling a biomolecule comprising a polymer backbone terminating into an end group capable of stoichiometrically binding to the biomolecule, and one or more pendant moieties each attached to the polymer backbone and capable of chelating with an element.

In some embodiments of the present disclosure, the tag is bio-compatible. In some embodiments, the polymer backbone of the tag is selected from the group consisting of polypeptide, polypeptoid, poly β-peptide, poly γ-peptide, poly δ-peptide, and a derivative thereof. In some embodiments, the polymer backbone is a homopolymer or a copolymer.

In some embodiments of the present disclosure, the polymer backbone is formed by polymerization of a monomer selected from the group consisting of NCA (N-carboxy anhydride), NTA (N-thiocarboxy anhydride), α-amino acid, β-amino acid, γ-amino acid, δ-amino acid, N-substituted amino acid, and a derivative thereof. In some embodiments, the N-substituted amino acid is N-substituted glycine.

In some embodiments of the present disclosure, the degree of polymerization of the polymer backbone is between 10 and 1000. In some embodiments, the degree of polymerization of the polymer backbone is between 50 and 300. In some embodiments, the polymer backbone has a polydispersity index (PDI) of less than 1.4.

In some embodiments of the present disclosure, the pendant moiety is capable of chelating with a metal or an isotope thereof. In some embodiments, the number of the pendant moieties attached to the polymer backbone is between 10 and 1000. In some embodiments, the number of the pendant moieties attached to the polymer backbone is between 50 and 300.

In some embodiments of the present disclosure, the pendant moiety is selected from the group consisting of EDTA, DTPA, DCTA, DOTA, TETA, NOTA and a derivative thereof. In some embodiments, the pendant moiety is selected from DOTA or DTPA.

In some embodiments of the present disclosure, each of the one or more pendant moieties is directly attached to the polymer backbone. In some embodiments of the present disclosure, each of the one or more pendant moieties is attached to the polymer backbone through a linker. In some embodiments of the disclosure, the linker comprises a 1,2,3-triazole group. In some embodiments, the linker is attached to the polymer backbone or the pendant moiety via a spacer. In some embodiments, the spacer is an alkyl group or a polyelthylene glycol (PEG) group.

In some embodiments of the present disclosure, the end group is attached to an N-terminal of the polymer backbone. In some embodiments, the end group comprises an azide-reactive group. In some embodiments, the azide-reactive group is a cyclooctyne or a derivative thereof.

In some embodiments of the present disclosure, the end group is attached to the polymer backbone via a spacer. In some embodiments, the spacer is an alkyl group or a polyelthylene glycol (PEG) group.

In yet another aspect, the disclosure provides a tag for labeling a biomolecule comprising a polymer backbone terminating into an end group capable of binding to the biomolecule, and one or more pendant moieties each attached to the polymer backbone and capable of chelating with an element, wherein the tag is bio-compatible.

In some embodiments, the polymer backbone of the tag is selected from the group consisting of polypeptide, polypeptoid, poly β-peptide, poly γ-peptide, poly δ-peptide, and a derivative thereof. In some embodiments, the polymer backbone is a homopolymer or a copolymer.

In some embodiments of the present disclosure, the polymer backbone is formed by polymerization of a monomer selected from the group consisting of NCA (N-carboxy anhydride), NTA (N-thiocarboxy anhydride), α-amino acid, β-amino acid, γ-amino acid, δ-amino acid, N-substituted amino acid, and a derivative thereof. In some embodiments, the N-substituted amino acid is N-substituted glycine.

In some embodiments of the present disclosure, the degree of polymerization of the polymer backbone is between 10 and 1000. In some embodiments, the degree of polymerization of the polymer backbone is between 50 and 300. In some embodiments, the polymer backbone has a polydispersity index (PDI) of less than 1.4.

In some embodiments of the present disclosure, the pendant moiety is capable of chelating with a metal or an isotope thereof. In some embodiments, the number of the pendant moieties attached to the polymer backbone is between 10 and 1000. In some embodiments, the number of the pendant moieties attached to the polymer backbone is between 50 and 300.

In some embodiments of the present disclosure, the pendant moiety is selected from the group consisting of EDTA, DTPA, DCTA, DOTA, TETA, NOTA and a derivative thereof. In some embodiments, the pendant moiety is selected from DOTA or DTPA.

In some embodiments of the present disclosure, each of the one or more pendant moieties is directly attached to the polymer backbone. In some embodiments of the present disclosure, each of the one or more pendant moieties is attached to the polymer backbone through a linker. In some embodiments of the disclosure, the linker comprises a 1,2,3-triazole group. In some embodiments, the linker is attached to the polymer backbone or the pendant moiety via a spacer. In some embodiments, the spacer is an alkyl group or a polyelthylene glycol (PEG) group.

In yet another aspect, the disclosure provides a tag for labeling a biomolecule comprising a polymer backbone terminating into an end group capable of binding to the biomolecule, and one or more pendant moieties each stoichiometrically attached to a repeating unit of the polymer backbone and capable of chelating with an element.

In some embodiments of the present disclosure, the pendant moiety is capable of chelating with a metal or an isotope thereof. In some embodiments, the number of the pendant moieties attached to the polymer backbone is between 10 and 1000. In some embodiments, the number of the pendant moieties attached to the polymer backbone is between 50 and 300.

In some embodiments of the present disclosure, the pendant moiety is selected from the group consisting of EDTA, DTPA, DCTA, DOTA, TETA, NOTA and a derivative thereof. In some embodiments, the pendant moiety is selected from DOTA or DTPA.

In some embodiments of the present disclosure, each of the one or more pendant moieties is directly attached to the polymer backbone. In some embodiments of the present disclosure, each of the one or more pendant moieties is attached to the polymer backbone through a linker. In some embodiments of the disclosure, the linker comprises a 1,2,3-triazole group. In some embodiments, the linker is attached to the polymer backbone or the pendant moiety via a spacer. In some embodiments, the spacer is an alkyl group or a polyelthylene glycol (PEG) group.

In another aspect, the disclosure provides an element tag for labeling a biomolecule comprising the tag of the disclosure as described above, wherein the pendant moiety attached to the polymer backbone chelates with an element.

In some embodiments of the present disclosure, the element is a metal or an isotope thereof. In some embodiments, the metal has a mass of more than 60. In some embodiments, the metal is selected from the group consisting of La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, Og and an isotope thereof. In some embodiments, the metal is a lanthanide metal or an isotope thereof. In some embodiments, the lanthanide metal is La, Lu, Pr, Nd, Pm, Sm, Eu, Gd, Tb, Dy, Ho, Er, Tm, Yb, Lu, or an isotope thereof.

In another aspect, the disclosure provides a conjugate for element analysis comprising a biomolecule coupled with the tag of the disclosure as described above.

In some embodiments, the biomolecule is pre-functionalized with a group suitable for covalently binding to the tag before coupling with the tag. In some embodiments, the biomolecule is pre-functionalized with one or more azide groups.

In some embodiments of the disclosure, the biomolecule is selected from the group consisting of peptide, protein, aptamer, antibody, enzyme, carbohydrate, nucleic acid, deoxyribonucleic acid, oligonucleotide, polypeptide, recombinant protein, ribonucleic acid lipid, and a derivative thereof. In some embodiments, the antibody is selected from a group consisting of monoclonal antibody, polyclonal antibody, antibody fragment, Fab fragment, Fc fragment, light chain, heavy chain, immunoglobin, and immunoglobin fragment.

In some embodiments, the conjugate further chelates with one or more elements. In some embodiments, the number of the elements chelating with the conjugate is between 10 and 1000. In some embodiments, the number of the elements chelating with the conjugate is between 50 and 300.

In some embodiments of the present disclosure, the element is a metal or an isotope thereof. In some embodiments, the metal has a mass of more than 60. In some embodiments, the metal is a lanthanide metal or an isotope thereof. In some embodiments, the lanthanide metal is La, Lu, Pr, Nd, Pm, Sm, Eu, Gd, Tb, Dy, Ho, Er, Tm, Yb, Lu, or an isotope thereof.

In another aspect, the disclosure relates to said conjugate for use in an element analysis. In some embodiments, the element analysis is MS. In some embodiments, the MS is ICP-MS or ICP-TOF-MS.

In another aspect, the disclosure provides a method for preparing the tag of the disclosure as described above, comprising providing a polymer backbone; attaching one or more pendant moieties capable of chelating with an element to the polymer backbone; and attaching an end group capable of binding to a biomolecule to one end of the polymer backbone.

In some embodiments, the polymer backbone is provided as a homopolymer or a copolymer. In some embodiments, the polymer backbone is provided by polymerization of 10-1000 monomers. In some embodiments, the polymer backbone is provided by polymerization of 50-300 monomers.

In some embodiments of the method, the polymer backbone is provided by polymerization of a monomer selected from the group consisting of NCA, NTA, α-amino acid, β-amino acid, γ-amino acid, δ-amino acid, N-substituted amino acid, and a derivative thereof. In some embodiments of the method, the N-substituted amino acid is N-substituted glycine.

In some embodiments of the method, the monomer is pre-functionalized before polymerization. In some embodiments, the monomer is pre-functionalized with an azide group. In some embodiments, the monomer is pre-functionalized with an alkynyl group.

In some embodiments of the method, the pendant moiety is selected from the group consisting of EDTA, DTPA, DCTA, DOTA, TETA, NOTA and a derivative thereof. In some embodiments of the method, the pendant moiety is DOTA or DTPA.

In some embodiments of the method, the pendant moiety is pre-functionalized with an azide group. In some embodiments of the method, the pendant moiety is pre-functionalized with an alkynyl group.

In some embodiments, the method of the disclosure further comprises protecting the pendant moiety before attaching the pendant moiety to the polymer backbone. In some embodiments, the pendant moiety is protected by a group selected from methyl ester, benzyl ester, tert-butyl ester, ester of 2, 6-disubstituted phenol, silyl ester, orthoester or oxazoline.

In some embodiments of the method, the pendant moiety is attached to the polymer backbone through a click reaction. In some embodiments, the click reaction is a copper-catalyzed click reaction.

In some embodiments, the method of the disclosure further comprises chelating an element with the pendant moiety before attaching the pendant moieties to the polymer backbone. In some embodiments, the method of the disclosure further comprises chelating an element with the pendant moiety after attaching the pendant moieties to the polymer backbone. In some embodiments, the method of the disclosure further comprises chelating an element with the pendant moiety of the tag before attaching the end group to the polymer backbone. In some embodiments, the method of the disclosure further comprises chelating an element with the pendant moiety of the tag after attaching the end group to the polymer backbone.

In some embodiments of the method, the number of the elements chelating with the pendant moiety is 10-1000. In some embodiments, the number of the elements chelating with the pendant moiety is 50-300.

In some embodiments, the method of the disclosure further comprises de-protecting the pendant moiety before chelating with the element.

In yet another aspect, the disclosure provides a method for preparing a conjugate for element analysis, comprising (i) pre-functionalizing a biomolecule; and (ii) contacting the tag of the disclosure with the biomolecule.

In some embodiments of the method for preparing the conjugate, the biomolecule is coupled with the end group of the tag through a copper-free click reaction. In some embodiments, the biomolecule is pre-functionalized with one or more azide groups.

In some embodiments, the biomolecule is selected from the group consisting of peptide, protein, aptamer, antibody, enzyme, carbohydrate, nucleic acid, deoxyribonucleic acid, oligonucleotide, polypeptide, recombinant protein, ribonucleic acid lipid, and a derivative thereof.

In some embodiments, the biomolecule is an antibody, and the pre-functionalization is performed by incorporating one or more GalNAz groups into one or more glycan chains of the antibody. In some embodiments, the pre-functionalization is performed by incorporating 4 GalNAz groups to the glycan chains of the antibody.

In some embodiments, the biomolecule is an oligonucleotide, and the pre-functionalization is performed by incorporating one or more azide-modified phosphoramidites into the oligonucleotide.

In still another embodiment, the biomolecule is a peptide, and the pre-functionalization is performed by incorporating one or more azide-modified amino acids into the peptide.

In some embodiments, the method for preparing the conjugate further comprises chelating an element with the conjugate. In some embodiments, the chelation is performed before coupling the tag with the biomolecule. In some embodiments, the chelation is performed after coupling the tag with the biomolecule.

In another aspect, the disclosure provides a method for quantifying an analyte in a sample, comprising (i) contacting the sample with a conjugate of the disclosure, wherein the biomolecule of the conjugate specifically binds to the analyte in the sample; and (ii) quantifying the analyte by determining the amount of the element in the conjugate through an element analysis. In some embodiments of the method for quantifying an analyte in a sample, the element analysis is performed with ICP-MS or ICP-TOF-MS.

In some embodiments, the biomolecule of the conjugate is further labelled with another tag. In some embodiments, the method for quantifying an analyte in a sample further comprises separating the conjugate binding to the analyte.

In some embodiments of the method for quantifying an analyte in a sample, the sample is obtained from a subject. In some embodiments, the sample is bodily fluid or tissue. In some embodiments, the bodily fluid is selected from the group consisting of whole blood, plasma, serum, urine, effusions, ascitic fluid, saliva, cerebrospinal fluid, cervical secretions, vaginal secretions, endometrial secretions, amniotic fluid, gastrointestinal secretions, bronchial secretions including sputum, breast fluid and secretions. In some embodiments, the tissue is a tumor tissue.

In some embodiments, the analyte is selected from the group consisting of cell, nucleic acid and protein. In some embodiments, the cell is a tumor cell.

In another aspect, the disclosure provides a kit comprising (i) the tag of the disclosure; and (ii) an instruction for using the kit. In some embodiments, the kit further comprises an azide reagent for pre-functionalizing a biomolecule. In some embodiments, the kit of the disclosure comprises the biomolecule. In some embodiments, the kit further comprises a catalyst for coupling the tag with the biomolecule. In some embodiments, the kit comprises a metal solution.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIGS. 1A-1C depict exemplary structures of the tag, the element tag and the conjugate of the present disclosure. FIG. 1A describes an exemplary tag of the disclosure wherein a polypeptide is used as the polymer backbone. At its N-terminal, the polypeptide was terminated into an end group capable of binding to a biomolecule. Pendant moieties are each attached to the repeating unit of the polymer backbone, and R is intended to comprise any structure between the pendant moiety and the polymer backbone. FIG. 1B describes an exemplary element tag of the disclosure comprising the tag of FIG. 1A, wherein the pendant moieties each chelates with an element. FIG. 1C describes an exemplary conjugate of the present disclosure, wherein the conjugate comprises a biomolecule covalently coupled with the element tag of FIG. 1B, and the biomolecule specifically binds to an analyte. The Conjugate may be subject to element analysis to detect the presence or amount of the analyte.

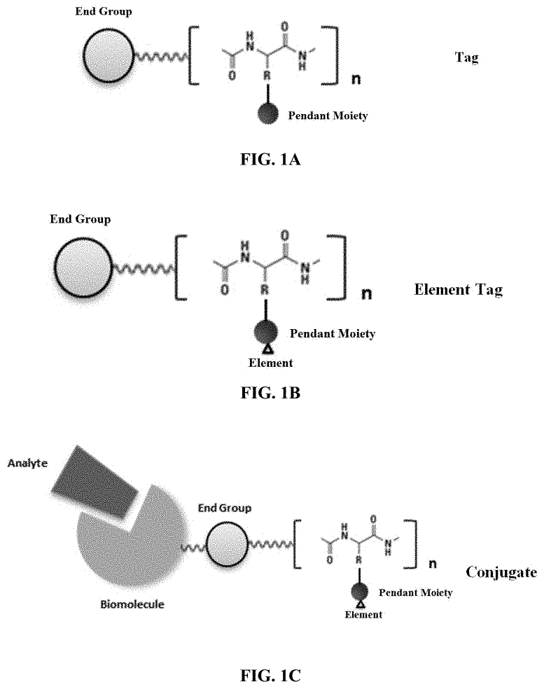

FIG. 2 depicts an exemplary scheme for synthesizing the tag of the present disclosure.

FIG. 3 exemplarily depicts labeling of IgG with the element tag of the present disclosure.

FIG. 4 exemplarily depicts quantification of the cell surface markers by using antibodies conjugated with the element tags of the present disclosure.

### DETAILED DESCRIPTION OF THE DISCLOSURE

The present disclosure recognizes the need for technologies for analysis of molecules based on element tags, where molecules to be analyzed are attached to an element first, and then subject to MS analysis. Moreover, the present disclosure recognizes the need for tags with improved biological compatibility, efficiency for quantification, and applicability in the clinical context. Finally, the present disclosure recognizes the need for improved tags and more effective method of use.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized. Unless defined otherwise, all technical and scientific terms used herein have the same meaning as commonly understood to one of ordinary skill in the art to which this disclosure belongs. Although any methods, devices, and materials similar or equivalent to those described herein may be used in the practice or testing of the disclosure, the preferred tags, conjugates, methods, and uses are now described.

As used herein and in the appended claims, the singular forms “a,” “and,” and “the” include plural referents unless the context clearly dictates otherwise. Thus, for example, reference to “a group” includes a plurality of such groups, reference to “an monomer” includes a plurality of such monomers, and reference to “the pendent moiety” includes reference to one or more pendant moieties (or to a plurality of pendant moieties) and equivalents thereof known to those skilled in the art, and so forth.

The term “about” when referring to a number or a numerical range means that the number or numerical range referred to is an approximation within experimental variability (or within statistical experimental error), and thus the number or numerical range may vary between 1% and 15% of the stated number or numerical range.

When ranges are used herein for physical properties, such as molecular weight, or chemical properties, such as chemical formulae, all combinations and subcombinations of ranges and specific embodiments therein are intended to be included. Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure provided herein.

The term “and/or” as used herein is a functional word to indicate that two words or expressions are to be taken together or individually. For example, A and/or B encompasses A alone, B alone, and A and B together.

The term “comprising” (and related terms such as “comprise” or “comprises” or “having” or “including”) is not intended to exclude that in other embodiments, for example, an embodiment of any composition of matter, composition, method, or process, or the like, described herein, may “consist of” or “consist essentially of” the described features.

As used herein, the term “tag” refers to a molecule that provides means for identifying and analyzing a target biomolecule to which it is attached. For example, a tag can comprise an element that permits identification, recognition, and/or molecular or biochemical manipulation of the target biomolecule to which it is attached. The process of attaching the tag to the biomolecule is sometimes referred to herein as “tagging” and a biomolecule that undergoes tagging or that contains a tag is referred to as “tagged” (e.g., “tagged biomolecule”).

The term “polymer” as used herein refers to a molecule (or macromolecule) composed of “repeating” monomeric units connected by covalent bonds. Any suitable polymer can be used to carry out the present disclosure. In some embodiments, the polymer of the present disclosure refers to a polymer consisting of identical monomer units, which is also referred as “homopolymer.” In some embodiments, the polymer of the present disclosure refers to a polymer consisting of two different types of monomers, or three different types of monomers, or more types of monomers distributed along the polymer backbone, which is also referred as “copolymer”. The polymers provided herein include, but are not limited to, linear polymers and branched polymers such as star polymers, comb polymers, brush polymers, ladders, and dendrimers. The term “backbone” as used herein refers to the structure comprising a polymer that optionally contains pendant groups.

The term “degree of polymerization” for the purpose of this disclosure refers to a number of monomers in a single polymer backbone. The term “polydispersity index (PDI)” for the purpose of this disclosure refers to a measure of the distribution of molecular mass in a given polymer sample, and it can be calculated by dividing the weight average molecular weight (M_{w}) by the number average molecular weight (Mₑ). As used herein, the term “weight average molecular weight” generally refers to a molecular weight measurement that depends on the contributions of polymer molecules according to their sizes. As used herein, the term “number average molecular weight” generally refers to a molecular weight measurement that is calculated by dividing the total weight of all the polymer molecules in a sample with the total number of polymer molecules in the sample. &M The term “pendant moiety” as used herein refers to a moiety covalently linked and pendant to the polymer backbone, which can also form a complex with an element. For example, the pendant moiety is capable of chelating with an element, including a metal or an isotope thereof. In particular, the pendant moiety may have at least two Lewis bases capable of making at least two simultaneous coordinate bonds with a transition metal ion. In some embodiments of this disclosure, the moiety is able to maintain its ability to form at least two coordinate bonds independent of its attachment to the backbone. A chelated metal is the metal ion coordinated or coordinately bonded to least two electron pairs of the pendant moiety. Usually, the electron pairs of a pendant moiety form coordinate bonds with a single metal ion; however, in some embodiments, the pendant moiety may form coordinate bonds with more than one metal ions, with a variety of binding modes being possible.

For the purpose of the present disclosure, the term “pendant group” is the same as “pendant moiety” and is a single pendant or terminal portion of the molecule containing two or more electron pairs that can be donated to metal ions. The pendant moiety of the backbone is expected to maintain its chelating function even it is detached from the backbone.

The term “linker” and “linker group” as used herein are molecules that link or bond two entities, for example, the polymer backbone and the pendant moiety, but are not a part of either of the individual linked entities. For the purpose of the present disclosure, the term “linker” and “linker group” are used interchangeably to refer to a moiety or group that are attachable to the polymer backbone on one end and the pendant group on the other end.

The term “stoichiometric” as used herein, refers to the ratio of the moles of reactants such as molecules or compounds participating in a chemical reaction being about 0.9 to about 1.1. “Stoichiometrically binding” as used herein means that the groups or moieties of the present disclosure react in a ratio from about 0.9:1 to about 1.1:1.

The term “end group” as used herein refers to any moiety or group that can be attached to a terminal of the polymer backbone so that to enable the polymer backbone binding to a biomolecule.

The term “element” as used herein refers to any chemical element which can chelate with the pendent moiety of the tag of the present disclosure. For example, the element can be a metal or an isotope thereof, including La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, Og, or an isotope thereof.

The term “conjugate” as used herein refers to a complex comprising the tag of the present disclosure and a biomolecule, where the tag and the biomolecule are connected together through a covalent bond. The term “covalent bond” refers to a bond between two atoms formed by sharing at least one pair of electrons and expressly excludes ionic bonds, hydrogen bonds, bonds formed by adsorption including chemical adsorption and physical adsorption, bonds formed from van der Waals bonds, and dispersion forces. In some embodiments of the present disclosure, the “covalent bond” between the tag and the biomolecule is formed between the end group of the tag and a functional group of the biomolecule.

The term “biomolecule” as used herein refers to any of a variety of biological molecules. Examples of the biomolecules include but are not limited to peptide, protein, aptamer, antibody, enzyme, carbohydrate, nucleic acid, deoxyribonucleic acid, oligonucleotide, polypeptide, recombinant protein, ribonucleic acid lipid, and a derivative thereof. More specifically, the term is intended to include, without limitation, RNA, DNA, oligonucleotides, modified or derivatized nucleotides, enzymes, receptors, receptor ligands (including hormones), antibodies, antigens, and toxins, as well as cells including blood cells and tissue cells.

The term “functional group,” “reactive group” and “reactive moiety” as used interchangeably herein, refer to specific substituents or moieties within molecules that are responsible for the characteristic chemical reactions of those molecules. The same functional group will undergo the same or similar chemical reaction(s) regardless of the size of the molecule it is a part of.

The term “azide-reactive group” as used herein, refers to a chemical moiety that reacts with an azide group to form a covalent bond. Examples of the azide-reactive groups include but are not limited to alkyne, phosphine (e.g. triaryl phosphine), cyclic alkyne such as cyclooctyne group.

The term “alkyne-reactive group” as used herein, refers to a chemical moiety that reacts with an alkyne group to form a covalent bond. Examples of alkyne-reactive groups include but are not limited to azide group.

The term “thiol-reactive group” as used herein, refers to a chemical moiety that reacts with a thiol group to form a covalent bond. Examples of thiol-reactive groups include but are not limited to alkene group and alkyne group.

The term “alkyne-reactive group” as used herein, refers to a chemical moiety that reacts with an alkyne group to form a covalent bond. Examples of alkyne-reactive groups include but are not limited to thiol group.

The term “alkene-reactive group” as used herein, refers to a chemical moiety that reacts with an alkene group to form a covalent bond. Examples of alkene-reactive groups include but are not limited to thiol group.

The term “tetrazine-reactive group” as used herein, refers to a chemical moiety that reacts with a tetrazine group to form a covalent bond. Examples of tetrazine-reactive groups include but are not limited to cyclooctene group.

The term “cyclooctene-reactive group” as used herein, refers to a chemical moiety that reacts with a cyclooctene group to form a covalent bond. Examples of cyclooctene-reactive groups include but are not limited to tetrazine group.

The term “MS” (Mass Spectrometry) is an analytical technique that ionizes chemical species and sorts the ions based on their mass-to-charge ratio. Various types of MS can be used according to the present disclosure, including but are not limited to matrix-assisted laser desorption/ionization source with a time-of-flight mass analyzer (MALDI-TOF), inductively coupled plasma-mass spectrometry (ICP-MS), accelerator mass spectrometry (AMS), atomic absorption spectroscopy (AAS), thermal ionization-mass spectrometry (TIMS), quadrupole mass analyzers, ion trap analyzers, orbitrap analyzers, electrospray ionization mass spectrometry (ESI), fourier transform mass spectrometry (e.g., Fourier transform ion cyclotron resonance), tandem mass spectrometry (MS/MS), liquid chromatography mass spectrometry (LC/MS), and spark source mass spectrometry (SSMS).

“ICP-MS” (Inductively Coupled Plasma Mass Spectrometry) is a type of mass spectrometry which is capable of detecting metals and several non-metals at concentrations as low as one part in 10¹⁵ (part per quadrillion, ppq) on non-interfered low-background isotopes. This is achieved by ionizing the sample with inductively coupled plasma and then using a mass spectrometer to separate and quantify those ions.

“ICP-TOF-MS” (Time of Flight Inductively Coupled Plasma-Mass Spectrometry) also provides high precision and reliable element trace analysis detection. The detection limit of the method ranges from the parts per million (ppm) (routine analysis for industrial production applications) to the parts per billion (ppb) or parts per trillion (ppt) levels for research and development purposes. It can also identify trace contamination and unknown chemical compounds.

ICP-MS and ICP-TOF-MS are applied to analyze proteins labelled with element tags. The element tag labelled proteins can be accurately quantified by ICP-MS or ICP-TOF-MS down to a lower amount which is at least 2-3 orders of magnitude more sensitive than other mass spectrometry based quantification methods. By using different elements, multiplexing can be used for the analysis of a plurality of proteins (proteomics) in biological sample.

The term “click reaction” refers to a class of biocompatible small molecule reactions commonly used in bio-conjugation, allowing the joining of substrates of choice with specific biomolecules. Click reaction is not a single specific reaction, but describes a way of generating products that follow examples in nature, which also generates substances by joining small modular units. The click chemistry approach was originally regarded as a method to rapidly generate complex substances by joining small subunits together in a modular fashion. (See, e.g., Kolb et al., 2004, Angew Chem Int Ed 40:3004-31; Evans, 2007, Aust J Chem 60:384-95.)

Various forms of click reactions are suitable for the purpose of the present disclosure, such as the Copper (I)-catalyzed azide-alkyne cycloaddition (CuAAC) (Tornoe et al., 2002, J Organic Chem 67:3057-64). Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiolyne reactions.

The Copper (I)-catalyzed azide-alkyne cycloaddition (CuAAC) uses a copper catalyst in the presence of a reducing agent to catalyze the reaction of a terminal alkyne group attached to a first molecule. In the presence of a second molecule comprising an azide moiety, the azide reacts with the activated alkyne to form a 1,4-disubstituted 1,2,3-triazole. The copper catalyzed reaction occurs at room temperature and is sufficiently specific that purification of the reaction product is often not required. (Rostovstev et al., 2002, Angew Chem Int Ed 41:2596; Tornoe et al., 2002, J Org Chem 67:3057.) The azide and alkyne functional groups are largely inert towards biomolecules in aqueous medium, allowing the reaction to occur in complex solutions. The triazole formed is chemically stable and is not subject to enzymatic cleavage, making the click chemistry product highly stable in biological systems.

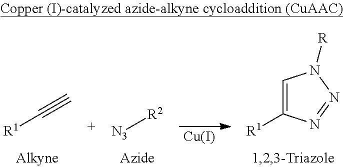

Thiol-ene and Thiol-yne reactions are click reactions involving the addition of an S—H bond across a double or triple bond, respectively. In some embodiments, the thiol-ene and/or the thiol-yne reactions are initiated by radical initiators and undergo a process with initiation, propagation, and termination steps. The thiol-ene reaction (also known as alkene hydrothiolation) is a reaction between a thiol and an alkene to form an alkyl sulfide. The thiol-yne reaction (also known as alkyne hydrothiolation) is a reaction between a thiol and an alkyne in place of the alkene in thiol-ene reaction to form an alkenyl sulfide.

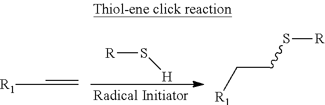

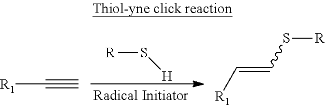

The “copper-free click reaction” has been applied for covalent modification of biomolecules in living systems. (See, e.g., Agard et al., 2004, J Am Chem Soc 126:15046-47.) In some embodiments, it uses ring strain in place of a catalyst to promote the reaction. In some embodiments, the copper-free click reaction includes [3+2] azide-alkyne cycloaddition and [4+2] Diels-Alder reaction.

For example, cyclooctyne has an 8-carbon ring structure comprising an internal alkyne bond, which can be used in [3+2] azide-alkyne cycloaddition. The closed ring structure induces a substantial bond angle deformation of the acetylene, which is highly reactive with azide groups to form a triazole. Similarly, cyclooctyne derivatives may be used for azide-alkyne cycloaddition, without the copper catalyst.

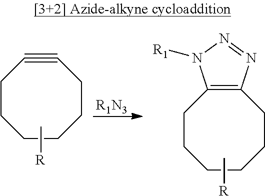

In a non-limiting [4+2] Diels-Alder reaction illustrated below, strained cyclooctenes or other activated alkenes react with tetrazines in an inverse electron-demand Diels-Alder to form a bicyclic intermediate which, upon the evolution of 1 equivalent of nitrogen, undergoes a retro-Diels-Alder reaction to afford the corresponding 4,5-dihydropyridazine compound without the need of a catalyst.

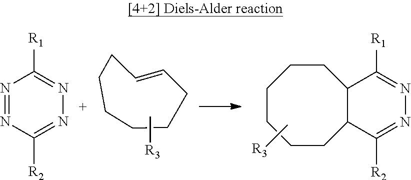

### Tag

In one aspect, disclosed herein is a tag for labeling a biomolecule comprising a polymer backbone terminating into an end group capable of binding to the biomolecule, one or more pendant moieties each attached to the polymer backbone and capable of chelating with an element.

Examples of polymers which can be used for the tag of the present disclosure include, but are not limited to, polycarboxylic acids, cellulosic polymers, proteins, polypeptides, polyvinylpyrrolidone, maleic anhydride polymers, polyamides, polyvinyl alcohols, polyethylene oxides, glycosaminoglycans, polysaccharides, polyesters, polyurethanes, polystyrenes, copolymers, silicones, polyorthoesters, polyanhydrides, copolymers of vinyl monomers, polycarbonates, polyethylenes, polypropylenes, polylactic acids, polyglycolic acids, polycaprolactones, polyhydroxybutyrate valerates, polyacrylamides, polyethers, polyurethane dispersions, polyacrylates, acrylic latex dispersions, polyacrylic acid and a derivative thereof.

The polymers of the present disclosure may be natural or synthetic in origin, including gelatin, chitosan, dextrin, cyclodextrin, poly(urethanes), poly(siloxanes), silicones, poly(acrylates) including poly(methyl methacrylate), poly(butyl methacrylate), and poly(2-hydroxy ethyl methacrylate), poly(vinyl alcohol) and a derivative thereof, and copolymers including those commonly sold as Teflon® products, poly(vinylidine fluoride), poly(vinyl acetate), poly(vinyl pyrrolidone), poly(acrylic acid), polyacrylamide, poly(ethylene-co-vinyl acetate), poly(ethylene glycol), poly(propylene glycol), poly(methacrylic acid), and derivatives thereof.

In some embodiments, polymers include, but are not limited to, absorbable and/or resorbable polymers including polylactides (PLA), polyglycolides (PGA), poly(lactide-co-glycolides) (PLGA), polyanhydrides, polyorthoesters, poly(N-(2-hydroxypropyl) methacrylamide), poly(1-aspartamide) and a derivative thereof.

In some embodiments, the present disclosure provides a tag for labeling a biomolecule comprising a polymer backbone terminating into an end group capable of binding to the biomolecule, and one or more pendant moieties each attached to the polymer backbone and capable of chelating with an element, wherein the tag is bio-compatible.

The term “bio-compatible” means that the substance will ultimately be “bio-absorbed” or cleared by the body with no adverse effects to the body. Similarly, the term “bioabsorbable” refers to a substance made from materials which undergo bioabsorption in vivo over a period of time such that long term accumulation of the material in the patient is reduced or avoided. Examples of bio-compatible polymers of the present disclosure include, but are not limited to, polypeptide, polypeptoid, poly β-peptide, poly γ-peptide, poly δ-peptide, and a derivative thereof.

In some embodiments, the polymer of the present disclosure is a homopolymer. In some embodiments, the polymer is a copolymer. In some embodiments, the polymer of the disclosure comprises two, three, four or more different polymers, such as two, three, four or more different homopolymers and/or copolymers.

The polymer backbone of the present disclosure can be formed by polymerization of a monomer. Examples of monomers that can be polymerized to form the polymer backbone of the present disclosure include, but are not limited to, carboxylic acids, cellulosic monomers, vinylpyrrolidones, maleic anhydrides, amides, vinyl alcohols, ethylene oxides, monosaccharides, esters, urethanes, styrenes, orthoesters, anhydrides, vinyl monomers, carbonates, ethylenes, propylenes, lactic acids, glycolic acids, caprolactones, hydroxybutyrate valerates, acrylamides, ethers, urethane dispersions, acrylates, acrylic latex dispersions, acrylic acids and a derivative thereof.

In some embodiments, monomers that can be polymerized to form the polymer backbone of the present disclosure include, but are not limited to, NCA (N-carboxy anhydride), NTA (N-thiocarboxy anhydride), α-amino acid, β-amino acid, γ-amino acid, δ-amino acid, N-substituted amino acid and a derivative thereof. For example, the N-substituted amino acid can be N-substituted glycine.

In some embodiments, monomers that can be polymerized to form the polymer backbone of the present disclosure include, but are not limited to, Alanine, Arginine, Asparagine, Asparagine, Cysteine, Glutamic acid, Glutamine, Glycine, Histidine, Isoleucine, Leucine, Lysine, Methionine, Phenylalanine, Proline, Serine, Threonine, Tryptophan, Tyrosine, Valine and a derivative thereof.

In some embodiments, monomers that can be polymerized to form the polymer backbone of the present disclosure include, but are not limited to, Ala-NCA, Arg-NCA, Asn-NCA, Asp-NCA, Cys-NCA, Glu-NCA, Gln-NCA, Gly-NCA, His-NCA, Ile-NCA, Leu-NCA, Lys-NCA, Met-NCA, Phe-NCA, Pro-NCA, Ser-NCA, Thr-NCA, Trp-NCA, Tyr-NCA, Tyr-NCA and a derivative thereof.

In some embodiments, the polymer backbone is a homopolymer polymerized by identical monomers. In some embodiments, the polymer backbone is a copolymer polymerized by different monomers.

As defined above, “degree of polymerization” means the number of monomers in the polymer backbone. In some embodiments of the present disclosure, the degree of polymerization of the polymer backbone is between 10 and 1000. In some embodiments, the degree of polymerization of the polymer backbone is between 20 and 1000. In some embodiments, the degree of polymerization of the polymer backbone is between 30 and 1000. In some embodiments, the degree of polymerization of the polymer backbone is between 40 and 1000. In some embodiments, the degree of polymerization of the polymer backbone is between 50 and 1000. In some embodiments, the degree of polymerization of the polymer backbone is between 100 and 1000. In some embodiments, the degree of polymerization of the polymer backbone is between 200 and 1000. In some embodiments, the degree of polymerization of the polymer backbone is between 50 and 900. In some embodiments, the degree of polymerization of the polymer backbone is between 50 and 800. In some embodiments, the degree of polymerization of the polymer backbone is between 50 and 700. In some embodiments, the degree of polymerization of the polymer backbone is between 50 and 600. In some embodiments, the degree of polymerization of the polymer backbone is between 50 and 500. In some embodiments, the degree of polymerization of the polymer backbone is between 50 and 400. In some embodiments, the degree of polymerization of the polymer backbone is between 50 and 300. In some embodiments, the degree of polymerization of the polymer backbone is between 50 and 200. In some embodiments, the degree of polymerization of the polymer backbone is between 50 and 100.

As defined above, “polydispersity index (PDI)” means a measure of the distribution of molecular mass in the polymer backbone. In some embodiments, the polymer backbone of the present disclosure has a PDI of less than 2.0. In some embodiments, the polymer backbone has a PDI of less than 1.9. In some embodiments, the polymer backbone has a PDI of less than 1.8. In some embodiments, the polymer backbone has a PDI of less than 1.7. In some embodiments, the polymer backbone has a PDI of less than 1.6. In some embodiments, the polymer backbone has a PDI of less than 1.5. In some embodiments, the polymer backbone has a PDI of less than 1.45. In some embodiments, the polymer backbone has a PDI of less than 1.4. In some embodiments, the polymer backbone has a PDI of less than 1.35. In some embodiments, the polymer backbone has a PDI of less than 1.3. In some embodiments, the polymer backbone has a PDI of less than 1.25. In some embodiments, the polymer backbone has a PDI of less than 1.2. In some embodiments, the polymer backbone has a PDI of less than 1.15. In some embodiments, the polymer backbone has a PDI of less than 1.1. In some embodiments, the polymer backbone has a PDI of less than 1.09. In some embodiments, the polymer backbone has a PDI of less than 1.08. In some embodiments, the polymer backbone has a PDI of less than 1.07. In some embodiments, the polymer backbone has a PDI of less than 1.06. In some embodiments, the polymer backbone has a PDI of less than 1.05. In some embodiments, the polymer backbone has a PDI of less than 1.04. In some embodiments, the polymer backbone has a PDI of less than 1.03. In some embodiments, the polymer backbone has a PDI of less than 1.02. In some embodiments, the polymer backbone has a PDI of less than 1.01. In some embodiments, the polymer backbone has a PDI of 1.

The “end group” of the tag of the present disclosure refers to any moiety or group that can be attached to a terminal of the polymer backbone to enable the polymer backbone binding to a biomolecule. In some embodiments, the end group is attached to an N-terminal of the polymer backbone. In some embodiments, the end group is attached to one or more N-terminals of the polymer backbone.

In some embodiments, the present disclosure provides a tag for labeling a biomolecule comprising a polymer backbone terminating into an end group capable of stoichiometrically binding to the biomolecule, and one or more pendant moieties each attached to the polymer backbone and capable of chelating with an element.

“Stoichiometrically” here means that the molar ratio of the end group to the reactive or functional group of the biomolecule is about 0.9 to about 1.1. In particular, in some embodiments, the molar ratio of the end group to the reactive or functional group of the biomolecule is about 0.9. In some embodiments, the molar ratio of the end group to the reactive or functional group of the biomolecule is about 0.91. In some embodiments, the molar ratio of the end group to the reactive or functional group of the biomolecule is about 0.92. In some embodiments, the molar ratio of the end group to the reactive or functional group of the biomolecule is about 0.93. In some embodiments, the molar ratio of the end group to the reactive or functional group of the biomolecule is about 0.94. In some embodiments, the molar ratio of the end group to the reactive or functional group of the biomolecule is about 0.95. In some embodiments, the molar ratio of the end group to the reactive or functional group of the biomolecule is about 0.96. In some embodiments, the molar ratio of the end group to the reactive or functional group of the biomolecule is about 0.97. In some embodiments, the molar ratio of the end group to the reactive or functional group of the biomolecule is about 0.98. In some embodiments, the molar ratio of the end group to the reactive or functional group of the biomolecule is about 0.99. In some embodiments, the molar ratio of the end group to the reactive or functional group of the biomolecule is about 1. In some embodiments, the molar ratio of the end group to the reactive or functional group of the biomolecule is about 1.01. In some embodiments, the molar ratio of the end group to the reactive or functional group of the biomolecule is about 1.02. In some embodiments, the molar ratio of the end group to the reactive or functional group of the biomolecule is about 1.03. In some embodiments, the molar ratio of the end group to the reactive or functional group of the biomolecule is about 1.04. In some embodiments, the molar ratio of the end group to the reactive or functional group of the biomolecule is about 1.05. In some embodiments, the molar ratio of the end group to the reactive or functional group of the biomolecule is about 1.06. In some embodiments, the molar ratio of the end group to the reactive or functional group of the biomolecule is about 1.07. In some embodiments, the molar ratio of the end group to the reactive or functional group of the biomolecule is about 1.08. In some embodiments, the molar ratio of the end group to the reactive or functional group of the biomolecule is about 1.09. In some embodiments, the molar ratio of the end group to the reactive or functional group of the biomolecule is about 1.1. It is to be understood that though a stoichiometric binding preferably have a molar ratio of about 0.9 to about 1.1, but a binding ratio below 0.8 and over 1.2 may also be regarded as a stoichiometrical binding. It is also to be understood that stoichiometrical binding does not mean that the molar ratio of the end group to the biomolecules is around 1:1, especially when there are more than one reactive or functional groups on one biomolecule.

In some embodiments, the end group comprises an azide-reactive group. Examples of azide-reactive groups of the present disclosure include, but are not limited to, alkynes and phosphines, for example, triaryl phosphine.

In some embodiments, the azide-reactive group comprises a cyclic alkyne, for example, a cyclooctyne or a derivative thereof. Examples of cyclooctynes or derivatives thereof of the present disclosure include, but are not limited to, DIFO (difluorinatedcyclooctynes), BCN (bicyclononyne), MAC (dibenzoazacyclooctyne), DIBO (dibenzocyclooctyne), ADIBO (azadibenzocyclooctyne) or a derivative thereof.

In some embodiments, the tag of the present disclosure comprises a polypeptide backbone terminated into a cyclooctyne, and the cyclooctyne includes but is not limited to DIFO, BCN, DIBAC, MO, ADIBO and/or a derivative thereof. In some embodiments, the tag of the present disclosure comprises a polypeptoid backbone terminated into a cyclooctyne, and the cyclooctyne includes but is not limited to DIFO, BCN, DIBAC, DB30, ADIBO and/or a derivative thereof. In some embodiments, the tag of the present disclosure comprises a poly β-peptide backbone terminated into a cyclooctyne, and the cyclooctyne includes but is not limited to DIFO, BCN, DIBAC, MO, ADIBO and/or a derivative thereof. In some embodiments, the tag of the present disclosure comprises a poly γ-peptide backbone terminated into a cyclooctyne, and the cyclooctyne includes but is not limited to DIFO, BCN, DIBAC, DB30, ADIBO and/or a derivative thereof. In some embodiments, the tag of the present disclosure comprises a poly δ-peptide backbone terminated into a cyclooctyne, and the cyclooctyne includes but is not limited to DIFO, BCN, DIBAC, MO, ADIBO and/or a derivative thereof.

In some embodiments, the end group comprises an alkyne-reactive group. Examples of alkyne-reactive groups include but are not limited to azide group and derivatives thereof.

In some embodiments, the end group can comprise a tetrazine-reactive group. Examples of tetrazine-reactive groups include but are not limited to cyclooctene group and derivatives thereof.

In some embodiments, the end group can comprise a cyclooctene-reactive group. Examples of cyclooctene-reactive groups include but are not limited to tetrazine group and derivatives thereof.

In some embodiments, the end group of the present disclosure is attached to the polymer backbone via a spacer. The term “spacer” or “spacer group” here refers to a molecular fragment which connects the end group to an end of the polymer backbone, for example, the N-terminal of the polymer backbone. Examples of spacers include but are not limited to polyester, polyamide, polyolefin, polyethylene oxides, glycosaminoglycans, polysaccharides, polyurethanes, polysulfone, polyester sulfone, polyphenyl ether, poly phenyl, polyetheretherketone, polyimide, polyetherimide, and any derivatives thereof. In some embodiments, the spacer can be selected from ester, amide, olefin, ethylene oxide, monosaccharide, urethanes, sulfone, ester sulfone, phenyl ether, phenyl, etheretherketone, imide, etherimide, and any derivative thereof. In some embodiments, the spacer is an alkyl group or a polyethylene glycol (PEG) group.

The pendant moiety of the tag of the present disclosure refers to a moiety covalently attached and pendant to the polymer backbone, and is capable of chelating with an element, including but not limited to, a metal or an isotope thereof. In some embodiments of this disclosure, the moiety is able to maintain its ability to form at least two coordinate bonds independent of its attachment to the backbone.

In some embodiments, the present disclosure provides a tag for labeling a biomolecule comprising a polymer backbone terminating into an end group capable of binding to the biomolecule, and one or more pendant moieties each stoichiometrically attached to a repeating unit of the polymer backbone and capable of chelating with an element.

“Stoichiometrically” here means that the molar ratio of the pendant moiety to the repeating unit of the polymer backbone is about 0.9 to about 1.1. In particular, in some embodiments, the molar ratio of the pendant moiety to the repeating unit of the polymer backbone is about 0.9. In some embodiments, the molar ratio of the pendant moiety to the repeating unit of the polymer backbone is about 0.91. In some embodiments, the molar ratio of the pendant moiety to the repeating unit of the polymer backbone is about 0.9. In some embodiments, the molar ratio of the pendant moiety to the repeating unit of the polymer backbone is about 0.91. In some embodiments, the molar ratio of the pendant moiety to the repeating unit of the polymer backbone is about 0.92. In some embodiments, the molar ratio of the pendant moiety to the repeating unit of the polymer backbone is about 0.93. In some embodiments, the molar ratio of the pendant moiety to the repeating unit of the polymer backbone is about 0.94. In some embodiments, the molar ratio of the pendant moiety to the repeating unit of the polymer backbone is about 0.95. In some embodiments, the molar ratio of the pendant moiety to the repeating unit of the polymer backbone is about 0.96. In some embodiments, the molar ratio of the pendant moiety to the repeating unit of the polymer backbone is about 0.97. In some embodiments, the molar ratio of the pendant moiety to the repeating unit of the polymer backbone is about 0.98. In some embodiments, the molar ratio of the pendant moiety to the repeating unit of the polymer backbone is about 0.99. In some embodiments, the molar ratio of the pendant moiety to the repeating unit of the polymer backbone is about 1. In some embodiments, the molar ratio of the pendant moiety to the repeating unit of the polymer backbone is about 1.01. In some embodiments, the molar ratio of the pendant moiety to the repeating unit of the polymer backbone is about 1.02. In some embodiments, the molar ratio of the pendant moiety to the repeating unit of the polymer backbone is about 1.03. In some embodiments, the molar ratio of the pendant moiety to the repeating unit of the polymer backbone is about 1.04. In some embodiments, the molar ratio of the pendant moiety to the repeating unit of the polymer backbone is about 1.05. In some embodiments, the molar ratio of the pendant moiety to the repeating unit of the polymer backbone is about 1.06. In some embodiments, the molar ratio of the pendant moiety to the repeating unit of the polymer backbone is about 1.07. In some embodiments, the molar ratio of the pendant moiety to the repeating unit of the polymer backbone is about 1.08. In some embodiments, the molar ratio of the pendant moiety to the repeating unit of the polymer backbone is about 1.09. In some embodiments, the molar ratio of the pendant moiety to the repeating unit of the polymer backbone is about 1.1. It is to be understood that though a stoichiometric binding preferably have a molar ratio of about 0.9 to about 1.1, but a binding ratio below 0.8 and over 1.2 may also be regarded as a stoichiometric binding.

Examples of the pendant moieties or pendant groups include, but are not limited to, EDTA (ethylenediaminetetraacetic acid), PDCA (2,6-pyridinedicarboxylic acid), DTPA (diethylenetriaminepentaacetic acid), DCTA (diaminocyclohexanetetraacetic Acid), DOTA (1,4,7,10-Tetraazacyclododecane-1,4,7,10-tetraacetic acid), TETA (N,N′-Bis(2-aminoethyl)ethane-1,2-diamine), NOTA (1,4,7-triazacyclononane-N,N′,N″-triacetic acid) and a derivative thereof. In some embodiments, the pendant moiety is DOTA or DTPA.

In some embodiments, the tag of the present disclosure comprises a polypeptide backbone terminated into a cyclooctyne including DIFO, BCN, DIBAC, DIBO, ADIBO and/or a derivative thereof, and one or more pendant moieties including EDTA, PDCA, DTPA, DCTA, DOTA, TETA, NOTA and/or a derivative thereof each stoichiometrically attached to the repeating unit of the polymer backbone. In some embodiments, the tag of the present disclosure comprises a polypeptoid backbone terminated into a cyclooctyne including DIFO, BCN, DIBAC, DIBO, ADIBO and/or a derivative thereof, and one or more pendant moieties including EDTA, PDCA, DTPA, DCTA, DOTA, TETA, NOTA and/or a derivative thereof each stoichiometrically attached to the repeating unit of the polymer backbone. In some embodiments, the tag of the present disclosure comprises a poly β-peptide backbone terminated into a cyclooctyne including DIFO, BCN, DIBAC, MO, ADIBO and/or a derivative thereof, and one or more pendant moieties including EDTA, PDCA, DTPA, DCTA, DOTA, TETA, NOTA and/or a derivative thereof each stoichiometrically attached to the repeating unit of the polymer backbone. In some embodiments, the tag of the present disclosure comprises a poly γ-peptide backbone terminated into a cyclooctyne including DIFO, BCN, DIBAC, DIBO ADIBO and/or a derivative thereof, and one or more pendant moieties including EDTA, PDCA, DTPA, DCTA, DOTA, TETA, NOTA and/or a derivative thereof each stoichiometrically attached to the repeating unit of the polymer backbone. In some embodiments, the tag of the present disclosure comprises a poly δ-peptide backbone terminated into a cyclooctyne including DIFO, BCN, DIBAC, DIBO, ADIBO and/or a derivative thereof, and one or more pendant moieties including EDTA, PDCA, DTPA, DCTA, DOTA, TETA, NOTA and/or a derivative thereof each stoichiometrically attached to the repeating unit of the polymer backbone.

In some embodiments, the tag of the present disclosure comprises a polymer backbone terminated into an end group capable of binding to the biomolecule and one or more EDTA each stoichiometrically attached to the repeating unit of the polymer backbone. In some embodiments, the tag of the present disclosure comprises a polymer backbone terminated into an end group capable of binding to the biomolecule and one or more PDCA each stoichiometrically attached to the repeating unit of the polymer backbone. In some embodiments, the tag of the present disclosure comprises a polymer backbone terminated into an end group capable of binding to the biomolecule and one or more DTPA each stoichiometrically attached to the repeating unit of the polymer backbone. In some embodiments, the tag of the present disclosure comprises a polymer backbone terminated into an end group capable of binding to the biomolecule and one or more DCTA each stoichiometrically attached to the repeating unit of the polymer backbone. In some embodiments, the tag of the present disclosure comprises a polymer backbone terminated into an end group capable of binding to the biomolecule and one or more DOTA each stoichiometrically attached to the repeating unit of the polymer backbone. In some embodiments, the tag of the present disclosure comprises a polymer backbone terminated into an end group capable of binding to the biomolecule and one or more TETA each stoichiometrically attached to the repeating unit of the polymer backbone. In some embodiments, the tag of the present disclosure comprises a polymer backbone terminated into an end group capable of binding to the biomolecule and one or more NOTA each stoichiometrically attached to the repeating unit of the polymer backbone.

The number of the pendant moieties attached to the polymer backbone of the present disclosure is between 10 and 1000. In some embodiments, the number of EDTA, PDCA, DTPA, DCTA, DOTA, TETA, NOTA and/or a derivative thereof attached to the polymer backbone of the present disclosure is between 10 and 1000. In some embodiments, the number of the pendant moieties attached to the polymer backbone of the present disclosure is between 20 and 1000. In some embodiments, the number of the pendant moieties attached to the polymer backbone of the present disclosure is between 30 and 1000. In some embodiments, the number of the pendant moieties attached to the polymer backbone of the present disclosure is between 40 and 1000. In some embodiments, the number of the pendant moieties attached to the polymer backbone of the present disclosure is between 50 and 1000. In some embodiments, the number of the pendant moieties attached to the polymer backbone of the present disclosure is between 100 and 1000. In some embodiments, the number of the pendant moieties attached to the polymer backbone of the present disclosure is between 50 and 900. In some embodiments, the number of the pendant moieties attached to the polymer backbone of the present disclosure is between 50 and 800. In some embodiments, the number of the pendant moieties attached to the polymer backbone of the present disclosure is between 50 and 700. In some embodiments, the number of the pendant moieties attached to the polymer backbone of the present disclosure is between 50 and 600. In some embodiments, the number of the pendant moieties attached to the polymer backbone of the present disclosure is between 50 and 500. In some embodiments, the number of the pendant moieties attached to the polymer backbone of the present disclosure is between 50 and 400. In some embodiments, the number of the pendant moieties attached to the polymer backbone of the present disclosure is between 50 and 300. In some embodiments, the number of the pendant moieties attached to the polymer backbone of the present disclosure is between 50 and 200. In some embodiments, the number of the pendant moieties attached to the polymer backbone of the present disclosure is between 50 and 100.

In some embodiments, the pendant moiety of the present disclosure is directly attached to the polymer backbone. In some embodiments of the present disclosure, the pendant moiety of the present disclosure is attached to the polymer backbone through a linker. Examples of the linkers include, but are not limited to, a 1,2,3-triazole moiety, an alkyl-sulfide moiety and an alkenyl-sulfide moiety.

In some embodiments, the linker is attached to the polymer backbone or the pendant moiety via a spacer. The term “spacer” or “spacer group” here refers to a molecular fragment which connects the linker to the polymer backbone or to the pendant moiety. In some embodiments, the spacer is an alkyl group or a polyelthylene glycol (PEG) group. In some embodiments, the spacer includes but is not limited to polyester, polyamide, polyolefin, polyethylene oxides, glycosaminoglycans, polysaccharides, polyurethanes, polysulfone, polyester sulfone, polyphenyl ether, poly phenyl, polyetheretherketone, polyimide, polyetherimide, and any derivatives thereof. In some embodiments, the spacer can be selected from ester, amide, olefin, ethylene oxide, monosaccharide, urethanes, sulfone, ester sulfone, phenyl ether, phenyl, etheretherketone, imide, etherimide, and any derivative thereof. In some embodiments, the spacers includes but is not limited to alkane, cycloalkane, alkene, cycloalkene, ether, thioether, ketone, sulfone, ester, thioester, amide, phenyl, pyridine, furan, thiophene, benzimidazole, benzoylpyridine, benzofuran, benzothiophene, biphenyl, naphthalene and any derivative thereof.

### Element Tag

In another aspect, the disclosure provides an element tag for labeling a biomolecule comprising the tag of the disclosure as described above, wherein the pendant moiety of the tag chelates with an element.

The term “element” as used herein refers to any chemical element which can chelate with the pendent moiety of the tag of the present disclosure. In some embodiments, the element is a metal or an isotope thereof. In some embodiments, the metal has a mass of more than 60. In some embodiments, the metal is selected from the group consisting of La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, Og and an isotope thereof.

The lanthanide metals are preferred due to their low abundancy in environment. In some embodiments, the metal is a lanthanide metal and is selected from La, Lu, Pr, Nd, Pm, Sm, Eu, Gd, Tb, Dy, Ho, Er, Tm, Yb, Lu, or an isotope thereof.

The number of the elements chelating with the tag of the present disclosure can be in the range of 10 to 1000. In some embodiments, the number of the elements chelating with the tag of the present disclosure is between 20 and 1000. In some embodiments, the number of the elements chelating with the tag of the present disclosure is between 30 and 1000. In some embodiments, the number of the elements chelating with the tag of the present disclosure is between 40 and 1000. In some embodiments, the number of the elements chelating with the tag of the present disclosure is between 50 and 1000. In some embodiments, the number of the elements chelating with the tag of the present disclosure is between 100 and 1000. In some embodiments, the number of the elements chelating with the tag of the present disclosure is between 50 and 900. In some embodiments, the number of the elements chelating with the tag of the present disclosure is between 50 and 800. In some embodiments, the number of the elements chelating with the tag of the present disclosure is between 50 and 700. In some embodiments, the number of the elements chelating with the tag of the present disclosure is between 50 and 600. In some embodiments, the number of the elements chelating with the tag of the present disclosure is between 50 and 500. In some embodiments, the number of the elements chelating with the tag of the present disclosure is between 50 and 400. In some embodiments, the number of the elements chelating with the tag of the present disclosure is between 50 and 300. In some embodiments, the number of the elements chelating with the tag of the present disclosure is between 50 and 200. In some embodiments, the number of the elements chelating with the tag of the present disclosure is between 50 and 100.

### Conjugate

In another aspect, the disclosure provides a conjugate for element analysis comprising a biomolecule coupled with the above described tag of the disclosure.

The term “conjugate” as used herein refers to a complex comprising the tag of the present disclosure and a biomolecule, and the tag and the biomolecule are connected together through a covalent bond. In some embodiments, the “covalent bond” between the tag and the biomolecule is formed by the end group of the tag and a functional group of the biomolecule.

The term “biomolecule” of the conjugate of present disclosure refers to any of a variety of biological materials. Examples of the biomolecules include, but are not limited to, peptide, protein, aptamer, antibody, enzyme, carbohydrate, nucleic acid, deoxyribonucleic acid, oligonucleotide, polypeptide, recombinant protein, ribonucleic acid lipid, and derivative thereof.

The term “antibody” or “antibodies” as used herein refers to immunoglobulin (Ig) molecules and antigen-binding portions of immunoglobulin molecules, i.e., molecules that contain an antigen binding site which specifically binds to an antigen. Structurally, a simple naturally occurring antibody (e.g., IgG) comprises four polypeptide chains: two heavy (H) chains and two light (L) chains that are inter-connected by disulfide bonds. The natural immunoglobulins represent a large family of molecules that include several types of molecules, including IgD, IgG, IgA, IgM and IgE.

“Antibodies” as used herein also refers to hybrid or altered antibodies. The antigen-binding function of an antibody can be performed by fragments of a naturally-occurring antibody, therefore, the term “antibodies” also encompasses fragments of antibodies, altered antibodies, or hybrid antibodies, including but are not limited to Fab fragment(s), and Fv fragments. These fragments are also known as “antigen-binding fragments”. Examples of binding fragments encompassed within the term “antigen-binding fragments” include but are not limited to (i) Fab fragments consisting of the VL, VH, CL and CHI domains; (ii) Fd fragments consisting of the VH and CHI domains; (iii) Fv fragments consisting of the VL and VH domains of a single arm of an antibody, (iv) dAb fragments which consists of a VH domain as described by (Ward et al, (1989) Nature 341:544-546), (v) isolated complementarity determining regions (CDRs); and (vi) F(ab′)2 fragments, which are bivalent fragments comprising two Fab fragments linked by a disulfide bridge at the hinge region. Furthermore, although the two domains of the Fv fragment are generally coded for by separate genes, a synthetic linker can be made that enables them to be made as a single protein chain (known as single chain Fv (scFv); Bird et al. (1988) Science 242:423-426; and Huston et al. (1988) PNAS 85:5879-5883) by recombinant methods. Such single chain antibodies are also encompassed within the term “antigen-binding fragments”. Preferably, the antibody fragments are those which are capable of crosslinking their target antigen, such as, e.g., bivalent fragments such as F(ab′)2 fragments. Alternatively, antibody fragments which do not themselves crosslink their target antigen (e.g., a Fab fragments) can be used in conjunction with secondary antibodies which serves to crosslink the antibody fragment, thereby crosslinking the target antigen.

In some embodiments, the “antibody” as used herein includes, but is not limited to, monoclonal antibody, polyclonal antibody, antibody fragment, Fab fragment, Fc fragment, light chain, heavy chain, immunoglobin, and immunoglobin fragment.

The “functional group” of the biomolecule refers to a specific substituent or moiety within the biomolecule that is responsible for the characteristic chemical reaction of the biomolecule. In some embodiments, the biomolecule of the disclosure may originally have the functional group suitable for covalently binding to the tag. In some embodiments, the biomolecule is pre-functionalized or modified with a functional group suitable for covalently binding to the tag before coupling with the tag. The biomolecule of the conjugate of the present disclosure may originally have one or more functional groups, or the biomolecule of the conjugate of the present disclosure may be modified with one or more functional groups. For example, the biomolecule of the conjugate of the present disclosure may originally have 1, 2, 3, 4 or more functional groups, or the biomolecule of the conjugate of the present disclosure may be modified with 1, 2, 3, 4 or more functional groups.

In some embodiments, the functional group of the biomolecule comprises an alkyne-reactive group. Examples of alkyne-reactive groups include azide group and derivative thereof.

In some embodiments, the functional group of the biomolecule comprises an azide-reactive group. Examples of azide-reactive groups include but are not limited to alkyne group such as cyclooctyne group and derivatives thereof.

In some embodiments, the functional group of the biomolecule comprises a tetrazine-reactive group. Examples of tetrazine-reactive groups include but are not limited to cyclooctene group and derivatives thereof.

In some embodiments, the functional group of the biomolecule comprises a cyclooctene-reactive group. Examples of cyclooctene-reactive groups include but are not limited to tetrazine group and derivatives thereof.

In some embodiments, the conjugate of the present disclosure further chelates with one or more elements. In some embodiments, the number of the elements chelating with the conjugate of the present disclosure is between 10 and 1000. In some embodiments, the number of the elements chelating with the conjugate of the present disclosure is between 20 and 1000. In some embodiments, the number of the elements chelating with the conjugate of the present disclosure is between 30 and 1000. In some embodiments, the number of the elements chelating with the conjugate of the present disclosure is between 40 and 1000. In some embodiments, the number of the elements chelating with the conjugate of the present disclosure is between 50 and 1000. In some embodiments, the number of the elements chelating with the conjugate of the present disclosure is between 100 and 1000. In some embodiments, the number of the elements chelating with the conjugate of the present disclosure is between 50 and 900. In some embodiments, the number of the elements chelating with the conjugate of the present disclosure is between 50 and 800. In some embodiments, the number of the elements chelating with the conjugate of the present disclosure is between 50 and 700. In some embodiments, the number of the elements chelating with the conjugate of the present disclosure is between 50 and 600. In some embodiments, the number of the elements chelating with the conjugate of the present disclosure is between 50 and 500. In some embodiments, the number of the elements chelating with the conjugate of the present disclosure is between 50 and 400. In some embodiments, the number of the elements chelating with the conjugate of the present disclosure is between 50 and 300. In some embodiments, the number of the elements chelating with the conjugate of the present disclosure is between 50 and 200. In some embodiments, the number of the elements chelating with the conjugate of the present disclosure is between 50 and 100.

In some embodiments, the element chelating with the conjugate is a metal or an isotope thereof. In some embodiments, the metal has a mass of more than 60. In some embodiments, the metal is selected from the group consisting of La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, Og and an isotope thereof.

In some embodiments, the metal is a lanthanide metal or an isotope thereof, for example, La, Lu, Pr, Nd, Pm, Sm, Eu, Gd, Tb, Dy, Ho, Er, Tm, Yb, Lu, or an isotope thereof.

In another aspect, the disclosure relates to said conjugate for use in an element analysis. In some embodiments, the element analysis is performed with MS. In some embodiment, the element analysis is performed with ICP-MS or ICP-TOF-MS.

### Applications

In another aspect, the disclosure provides a method for quantifying an analyte in a sample, comprising (i) contacting the sample with a conjugate of the disclosure, wherein the biomolecule of the conjugate specifically binds to the analyte in the sample; and (ii) quantifying the analyte by determining the amount of the element in the conjugate through an element analysis. In some embodiments, the element analysis is performed with ICP-MS or ICP-TOF-MS.

Another aspect of the disclosure is to provide a method for multiplex analysis of two or more analytes in a sample, comprising: (i) contacting the sample with two or more conjugates of the disclosure, wherein the biomolecules of the two or more conjugates specifically bind to two or more analytes in the sample; and (ii) analyzing the two or more analytes binding to the two or more conjugates through an element analysis.

The term “sample” as used herein refers to any sample comprising the analyte to be quantified by the method of the disclosure. For example, the sample can be a biological sample obtained from a subject, including bodily fluid or tissue. In some embodiments, the bodily fluid can be whole blood, plasma, serum, urine, effusions, ascitic fluid, saliva, cerebrospinal fluid, cervical secretions, vaginal secretions, endometrial secretions, amniotic fluid, gastrointestinal secretions, bronchial secretions including sputum, breast fluid and/or secretions collected from the subject. In some embodiments, the tissue can be breast tissue, uterine tissue, cervical tissue, intestinal tissue, colorectal tissue, esophageal tissue, prostate tissue, lung tissue, heart tissue, muscle tissue, skin tissue, kidney tissue, cornea tissue, liver tissue, lymph tissue, brain tissue, connective tissue, soft tissue, epithelial tissue, endothelial tissue and bone. In some embodiments, the tissue is a tumor tissue. In some embodiments, the sample is a tumor tissue from breast, uterine, cervix, intestine, colon, esophagus, prostate, lung, heart, muscle, skin, kidney, liver, lymph node, brain and bone. In some embodiments, the analyte is selected from the group consisting of cell, nucleic acid and protein. In some embodiments, the cell is a tumor cell.

In some embodiments, the biomolecule is an antibody, and the analyte is a molecule that specifically binds with the antibody, such as an antigen. Through the method of the present disclosure, the antigen can be quantified by determining the amount of the element in the antibody conjugate through an element analysis, such as through ICP-MS or ICP-TOF-MS.

In some embodiments, the biomolecule of the conjugate is further labelled with another tag, including His tag, HA tag, ERK tag, GFP tag, Myc tag, FLAG tag, GST tag, Strep tag, β-Gal tag and/or MBP tag. Such tag, in contrast to the tag of the present disclosure, is called “another tag” or “the other tag”, which may be used to isolate, purify, detect or analyze the biomolecule of the conjugate.

In some embodiments, the method for quantifying an analyte in a sample further comprises isolating the conjugate binding to the analyte, for example, by the other tag of the biomolecule.

The tag or conjugate of the disclosure described herein can be used in a wide variety of applications including, medical diagnostics, medical prognostics, biological research, and water and soil testing. Similarly, the tag or conjugate of the disclosure may be used to detect a wide variety of analytes, including cells, microbes, bacteria, viruses, proteins, peptides, carbohydrates, nucleic acids or portions thereof.

The tag or conjugate of the disclosure are also useful for many detection and/or imaging applications. The detection and/or imaging applications include, but are not limited to, single-cell labeling, multi-cell labeling, tissue labeling, organ labeling, in vitro labeling, and in vivo labeling. The detection and/or imaging of cells may include detection and/or imaging of molecules expressed by the cells, such as, extracellular molecules or intracellular molecules. The detection and/or imaging of cells may include detection and/or imaging of molecules attached to the cells such as proteins, sugars, particulates.

In some embodiments, the present disclosure provides methods for using the tag or conjugate of the disclosure to detect analytes within a sample such as a mixed sample. In some embodiments, the sample may be a fluid sample. The fluid sample may be a biological fluid sample, for example a blood sample, plasma sample, saliva sample, urine sample, lymph sample, or spinal fluid sample. In some embodiments, the sample may be an environmental fluid sample, for example from a lake, river, ocean, pond, stream, spring, marsh, or reservoir. In some embodiments, the sample may be a water sample, for example from a desalinization plant, water treatment plant, reservoir, spring, stream, glacial water flow, water tower, or other water source that may be contemplated as a source of potable water.

In some embodiments, a molecule expressed by an analyte such as a cell may be detected with the tag or conjugate of the disclosure provided herein. For example, cells may be contacted with the conjugate of the disclosure with a biomolecule (e.g., antibody) that recognizes another molecule of the cell (e.g., cell surface marker, intracellular marker, etc.). Non-limiting examples of cells include: mammalian cells, human cells, non-human mammalian cells, eukaryotic cells, prokaryotic cells, animal cells, insect cells, bacteria cells, microbial cells, fungal cells, amphibian cells and fish cells. The cells can originate from a variety of tissues including but are not limited to: neural crest tissue, endodermal tissue, ectodermal tissue, mesodermal tissue, and mesenchymal tissue. Cell types may include but are not limited to: breast cells, brain cells, neural cells, pancreatic cells, liver cells, gall bladder cells, gastrointestinal cells, stomach cells, kidney cells, cells of the reproductive system, heart cells, skin cells, colon cells, urethral cells, endodermal cells, muscle cells, fibroblasts, adipocytes, tumor cells, cancer cells, virally-infected cells, bacterial infected cells, stem cells, dividing cells, apoptotic cells, necrotic cells, blood cells, white blood cells, and stromal cells.

In some embodiments, the cell may express an antigen that may be detected by the conjugate. For example, the biomolecule may be an antibody, and the antibody may be specific for EpCAM which is expressed on some cancerous cells, including MCF-7 cells. Other examples of antibodies that may be conjugated to the tag include but are not limited to the pan-cytokeratin antibody A45B/B3, AE1/AE3, CAM5.2 (pan-cytokeratin antibodies that recognize Cytokeratin 8 (CK8), Cytokeratin 18 (CK18), or Cytokeratin 19 (CK19)) and ones against breast cancer antigen NY-BR-1 (also known as B726P, ANKRD30A, Ankyrin repeat domain 30A); B305D isoform A or C (B305D-A to B305D-C; also known as antigen B305D); Hermes antigen (also known as Antigen CD44, PGP1); E-cadherin (also known as Uvomorulin, Cadherin-1, CDH1); Carcino-embryonic antigen (CEA; also known as CEACAM5 or Carcino-embryonic antigen-related cell adhesion molecule 5); β-Human chorionic gonadotophin (β-HCG; also known as CGB, chronic gonadotrophin, (3 polypeptide); Cathepsin-D (also known as CTSD); Neuropeptide Y receptor Y3 (also known as NPY3R; Lipopolysaccharide-associated protein3, LAP3, Fusion; chemokine (CXC motif, receptor 4); CXCR4; Oncogene ERBB1 (also known as c-erbB-1, Epidermal growth factor receptor, EGFR); Her-2 Neu (also known as c-erbB-2 or ERBB2); GABA receptor A, pi (π) polypeptide (also known as GABARAP, GABA-A receptor, pi (π) polypeptide (GABA A(π), γ-Aminobutyric acid type A receptor pi (it) subunit), or GABRP); ppGalNac-T(6) (also known as β-1-4-N-acetyl-galactosaminyl-transferase 6, GalNActransferase 6, GalNAcT6, UDP-N-acetyl-d-galactosamine:polypeptide N-acetylgalactosaminyltransferase 6, or GALNT6); CK7 (also known as Cytokeratin 7, Sarcolectin, SCL, Keratin 7, or KRT7); CK8 (also known as Cytokeratin 8, Keratin 8, or KRT8); CK18 (also known as Cytokeratin 18, Keratin 18, or KRT18); CK19 (also known as Cytokeratin 19, Keratin 19, or KRT19); CK20 (also known as Cytokeratin 20, Keratin 20, or KRT20); Mage (also known as Melanoma antigen family A subtytpes or MAGE-A subtypes); Mage3 (also known as Melanoma antigen family A 3, or MAGA3); Hepatocyte growth factor receptor (also known as HGFR, Renal cell carninoma papillary 2, RCC P2, Protooncogene met, or MET); Mucin-1 (also known as MUC1, Carcinoma Antigen 15.3, (CA15.3), Carcinoma Antigen 27.29 (CA 27.29); CD227 antigen, Episialin, Epithelial Membrane Antigen (EMA), Polymorphic Epithelial Mucin (PEM), Peanut-reactive urinary mucin (PITM), Tumor-associated glycoprotein 12 (TAG12)); Gross Cystic Disease Fluid Protein (also known as GCDFP-15, Prolactin-induced protein, PIP); Urokinase receptor (also known as uPR, CD87 antigen, Plasminogen activator receptor urokinase-type, PLAUR); PTHrP (parathyrold hormone-related proteins; also known as PTHLH); BS106 (also known as B511S, small breast epithelial mucin, or SBEM); Prostatein-like Lipophilin B (LPB, LPHB; also known as Antigen BU101, Secretoglobin family 1-D member 2, SCGB1-D2); Mammaglobin 2 (MGB2; also known as Mammaglobin B, MGBB, Lacryglobin (LGB) Lipophilin C (LPC, LPHC), Secretoglobin family 2A member 1, or SCGB2A1); Mammaglobin (MGB; also known as Mammaglobin 1, MGB1, Mammaglobin A, MGBA, Secretoglobin family 2A member 2, or SCGB2A2); Mammary serine protease inhibitor (Maspin, also known as Serine (or Cysteine) proteinase inhibitor Glade B (ovalbumin) member 5, or SERPINB5); Prostate epithelium-specific Ets transcription factor (PDEF; also known as Sterile alpha motif pointed domain-containing ets transcription factor, or SPDEF); Tumor-associated calcium signal transducer 1 (also known as Colorectal carcinoma antigen CO17-1A, Epithelial Glycoprotein 2 (EGP2), Epithelial glycoprotein 40 kDa (EGP40), Epithelial Cell Adhesion Molecule (EpCAM), Epithelial-specific antigen (ESA), Gastrointestinal tumor-associated antigen 733-2 (GA733-2), KS1/4 antigen, Membrane component of chromosome 4 surface marker 1 (M4S1), MK-1 antigen, MIC18 antigen, TROP-1 antigen, or TACSTD1); Telomerase reverse transcriptase (also known as Telomerase catalytic subunit, or TERT); Trefoil Factor 1 (also known as Breast Cancer Estrogen-Inducible Sequence, BCEI, Gastrointestinal Trefoil Protein, GTF, pS2 protein, or TFF1); folate; or Trefoil Factor 3 (also known as Intestinal Trefoil Factor, ITF, p1.B; or TFF3).

In some embodiments, the method provided herein comprises incubating the tag or conjugate of the present disclosure. For example, the tag of the disclosure may be incubated with the biomolecule (such as antibodies); the conjugates (including the biomolecule conjugated to the tag) may be incubated with an analyte (e.g., cells). The incubation may last for less than or equal to 100 hours, 75 hours, 60 hours, 50 hours, 24 hours, 20 hours, 15 hours, 10 hours, 5 hours, 3 hours, 2 hours, or 1 hour. In some embodiments, the incubation may be greater than 5 minutes, 10 minutes, 30 minutes, 1 hour, 2 hours, 5 hours, 10 hours, 24 hours, 30 hours, 50 hours, 60 hours, 75 hours or 100 hours. In some embodiments, the incubation may be 5 minutes, 10 minutes, 30 minutes, 1 hour, 2 hours, 5 hours, 10 hours, 24 hours, 30 hours, 50 hours, 60 hours, 75 hours or 100 hours. In some embodiments, the incubation may be about 5 minutes, 10 minutes, 30 minutes, 1 hour, 2 hours, 5 hours, 10 hours, 24 hours, 30 hours, 50 hours, 60 hours, 75 hours or 100 hours.

In some embodiments, a sample comprising an analyte is pre-treated for detection. At any stage of a method provided herein, the analyte (e.g., cells) may be incubated with a blocking buffer to prevent or reduce non-specific binding of the biomolecule of the conjugate.

At any stage of a method provided herein, the analytes (e.g., cells) may be washed with a suitable buffer solution. At any stage of a method provided herein, the analytes (e.g., cells) may be concentrated with a suitable method. For example, the cells may be concentrated by, for example, centrifugation or filtration.

In some embodiments, prior to detection using the tag or conjugate of the disclosure, analytes can be isolated by a chromatography method, a filtration method, a capillary electrophoresis method, a gel electrophoresis method, a liquid extraction method, a precipitation method, or an immunoprecipitation method. In some embodiments, a plurality of assays can be performed in parallel to improve analysis throughput.

In some embodiments, the present disclosure provides a method for detecting an analyte, and the method comprises: isolating the analyte from a mixture; contacting the isolated analyte with a solution comprising the tag conjugated to a biomolecule specific for the isolated analyte; and detecting the amount of the tag to obtain the amount of the analyte. In some embodiments, the present methods quantitate an analyte.

In some embodiments, analysis is performed on a sample from one or more biological cells, tissues, fluids, or samples. In some embodiments, the analysis can be performed on a sample after it has been collected from cells, tissues, fluids or samples. In some embodiments, analysis can be performed on a sample that comprises multiplex analytes. In some embodiments, analysis can be performed on a purified analyte.

### Method

### Preparation of the Tag

In another aspect, the disclosure relates to a method for preparing the tag of the present disclosure, comprising providing a polymer backbone; attaching one or more pendant moieties capable of chelating with an element to the polymer backbone; and attaching an end group capable of binding to a biomolecule to one end of the polymer backbone.

In some embodiments, the polymer backbone is provided as a homopolymer or a copolymer. In particular, the polymer backbone may be a homopolymer synthesized by identical monomers, or a copolymer synthesized by different monomers. Any suitable polymers can be used to carry out the present disclosure.

Monomers that can be polymerized to form the polymer backbone include, but are not limited to, carboxylic acids, cellulosic monomers, vinylpyrrolidones, maleic anhydrides, amides, vinyl alcohols, ethylene oxides, monosaccharides, esters, urethanes, styrenes, orthoesters, anhydrides, vinyl monomers, carbonates, ethylenes, propylenes, lactic acids, glycolic acids, caprolactones, hydroxybutyrate valerates, acrylamides, ethers, urethane dispersions, acrylates, acrylic latex dispersions, acrylic acid, and a derivative thereof.

In some embodiments, monomers that can be polymerized to form the polymer backbone include, but are not limited to, NCA (N-carboxy anhydride), NTA (N-thiocarboxy anhydride), α-amino acid, β-amino acid, γ-amino acid, δ-amino acid, N-substituted amino acid, and a derivative thereof. For example, N-substituted amino acid that can be polymerized to form the polymer backbone is N-substituted glycine.

In some embodiments, monomers that can be polymerized to form the polymer backbone include, but are not limited to, Alanine, Arginine, Asparagine, Asparagine, Cysteine, Glutamic acid, Glutamine, Glycine, Histidine, Isoleucine, Leucine, Lysine, Methionine, Phenylalanine, Proline, Serine, Threonine, Tryptophan, Tyrosine, Valine, and a derivative thereof. In some embodiments, monomers that can be polymerized to form the polymer backbone include but are not limited to Ala-NCA, Arg-NCA, Asn-NCA, Asp-NCA, Cys-NCA, Glu-NCA, Gln-NCA, Gly-NCA, His-NCA, Ile-NCA, Leu-NCA, Lys-NCA, Met-NCA, Phe-NCA, Pro-NCA, Ser-NCA, Thr-NCA, Trp-NCA, Tyr-NCA, Tyr-NCA and a derivative thereof.

In some embodiments, the polymer backbone is provided by polymerization of 10-1000 monomers. For example, the polymer backbone is provided by polymerization of 20-1000 monomers, 30-1000 monomers, 40-1000 monomers, 50-1000 monomers, 100-1000 monomers, 200-1000 monomers, 50-900 monomers, 50-800 monomers, 50-700 monomers, 50-600 monomers, 50-500 monomers, 50-400 monomers, 50-300 monomers, 50-200 monomers or 50-100 monomers.

In some embodiments of the method, the monomer is pre-functionalized before polymerization. The terms “functionalization” and “functionalize” mean modifying a molecule with a “functional group,” “reactive group” or “reactive moiety”. The monomer may be functionalized so that the pendant group can attach to the monomer. The functionality introduced to the monomer is compatible with polymerization conditions.

In some embodiments of the disclosure, the monomer is pre-functionalized with an azide-reactive group, for example, with an alkynyl group. In some embodiments of the method, the monomer is pre-functionalized with an alkynyl-reactive group, for example, with an azide group. In some embodiments, the monomer is pre-functionalized into a precursor so that the precursor can be further functionalized with the azide-reactive group, for example, with an alkynyl group, or functionalized with the alkynyl-reactive group, for example, with an azide group. In some embodiments, the monomer is pre-functionalized with a thiol-reactive group, for example, with an alkene group or an alkyne group. In some embodiments, the monomer is pre-functionalized with an alkene-reactive group or an alkyne-reactive group, for example, with a thiol group.

In some embodiments, the pendant moiety attaching to the monomer or the repeating unit of the polymer backbone is selected from the group consisting of EDTA, DTPA, DCTA, DOTA, TETA, NOTA and a derivative thereof. In some embodiments, the pendant moiety is DOTA or DTPA.

In some embodiments, the pendant moiety is pre-functionalized to attach to the monomer or the repeating unit of the polymer backbone. In some embodiments, the pendant moiety is pre-functionalized with an azide-reactive group, for example, with an alkynyl group. In some embodiments of the method, the pendant moiety is pre-functionalized with an alkynyl-reactive group, for example, with an azide group. In some embodiments, the pendant moiety is pre-functionalized with a thiol-reactive group, for example, with an alkene group or an alkyne group. In some embodiments, the pendant moiety is pre-functionalized with an alkene-reactive group or an alkyne-reactive group, for example, with a thiol group.

In some embodiments, the pendant moiety of the present disclosure is stoichiometrically attached to the monomer or repeating unit of the polymer backbone, for example, through a click reaction.

In some embodiments, the “click reaction” between the pendant moiety and the monomer or the repeating unit of the polymer backbone refers to the Huisgen cycloaddition between an azide and an alkynyl, which forms a 1,2,4-triazole, and the click reaction is catalyzed by a copper catalyst.

In some embodiments, the copper catalyst is selected from the group consisting of copper nitrate, copper formate, copper nitrite, copper nitride, copper cyanide, copper ferrocyanide, copper chloride, copper bromide, copper perchlorate, copper bromate, copper iodide, copper sulfide, copper sulfate, copper thiocyanate, copper carbonate, copper acetate, copper oxalate, copper butyrate, copper citrate, copper benzoate, copper borate, copper phosphate, copper carbide, copper chromate, copper tungstate, and any mixture thereof.

In some embodiments, the copper catalyst is selected from the group consisting of cuprous nitrate, cuprous formate, cuprous nitrite, cuprous nitride, cuprous cyanide, cuprous ferrocyanide, cuprous chloride, cuprous bromide, cuprous perchlorate, cuprous bromate, cuprous iodide, cuprous sulfide, cuprous sulfate, cuprous thiocyanate, cuprous carbonate, cuprous acetate, cuprous oxalate, cuprous butyrate, cuprous citrate, cuprous benzoate, cuprous borate, cuprous phosphate, cuprous carbide, cuprous chromate, cuprous tungstate, and any mixture thereof.

In some embodiments, the method for preparing the tag of the disclosure may further comprises protecting the pendant moiety before attaching the pendant moiety to the monomer or the repeating unit of the polymer backbone.

The term “protecting,” as used herein, refers to adding a “protecting” group or moiety that prevents reaction of the chemically reactive functional group under certain reaction conditions. The protecting group may vary depending on the type of chemically reactive group being protected. For example, the pendant moiety of the disclosure can be protected by a group selected from methyl ester, benzyl ester, tert-butyl ester, ester of 2,6-disubstituted phenol, silyl ester, orthoester or oxazoline.

In some embodiments, the method for preparing the tag of the disclosure further comprises attaching an end group capable of binding to a biomolecule to one end of the polymer backbone. In some embodiments, the end group is attached to an N-terminal of the polymer backbone. In some embodiments, one or more end groups may be attached to one or more ends of the polymer backbone.

In some embodiments, the end group comprises an azide-reactive group such as a cyclooctyne or a derivative thereof. Examples of the azide-reactive group include but are not limited to DIFO, BCN, DIBAC, DIBO, ADIBO and derivatives thereof.

In some embodiments, the end group comprises an alkyne-reactive group. Examples of alkyne-reactive groups include but are not limited to azide group and derivatives thereof.

In some embodiments, the end group comprises a tetrazine-reactive group. Examples of tetrazine-reactive groups include but are not limited to cyclooctene group and derivatives thereof.

In some embodiments, the end group comprises a cyclooctene-reactive group. Examples of cyclooctene-reactive groups include but are not limited to tetrazine group and derivatives thereof.

In some embodiments, the end group is attached to the end of the polymer backbone through a spacer, and examples of the spacers include, but are not limited to, an alkyl group or a polyethylene glycol (PEG) group.

In some embodiments, the method of the disclosure further comprises chelating an element with the pendant moiety. In some embodiments, the element is a metal or an isotope thereof. In some embodiments, the metal has a mass of more than 60. In some embodiments, the metal is selected from the group consisting of La, Lu, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, At, Rn, Fr, Ra, Ac, Th, Pa, U, Np, Pu, Am, Cm, Bk, Cf, Es, Fm, Md, No, Lr, Rf, db, Sg, Bh, Hs, Mt, Ds, Rg, Cn, Nh, Fl, Mc, Lv, Ts, Og and an isotope thereof.

In some embodiments, the metal is a lanthanide metal and is selected from La, Lu, Pr, Nd, Pm, Sm, Eu, Gd, Tb, Dy, Ho, Er, Tm, Yb, Lu, or an isotope thereof. In some embodiments, the number of the elements chelating with the pendant moiety of the tag is 10-1000. In some embodiments, the number of the elements chelating with the pendant moiety is 50-300.

In some embodiments of the method for preparing the tag of the present disclosure, the element chelates with the pendant moiety before attaching the pendant moiety to the monomer or the repeating unit of the polymer backbone. In some embodiments, the element chelates with the pendant moiety after attaching the pendant moiety to the monomer or the repeating unit of the polymer backbone. In some embodiments, the element chelates with the pendant moiety of the tag before attaching the end group to the polymer backbone. In some embodiments, the element chelates with the pendant moiety of the tag after attaching the end group to the polymer backbone. In some embodiments, the method of the disclosure further comprises de-protecting the pendant moiety before chelating with the element.

In some embodiments of the method for preparing the tag of the present disclosure, the pendant moiety chelates with the element before being attached to the monomer or repeating unit of the polymer backbone. In some embodiments, the pendant moiety chelates with the element after being attached to the monomer or repeating unit of the polymer backbone. In some embodiments, the method further comprises de-protecting the pendant moiety before chelating with the element. In some embodiments, the pendant moiety is attached to the monomer before monomer polymerization. In some embodiments, the pendant moiety is attached to the repeating unit of the polymer backbone after monomer polymerization. In some embodiments, the pendant moiety is attached to the repeating unit of the polymer backbone before attaching the end group to an end of the polymer backbone. In some embodiments, the pendant moiety is attached to the repeating unit of the polymer backbone after attaching the end group to an end of the polymer backbone.

In some embodiments of the method for preparing the tag of the present disclosure, the monomer is attached with the pendant moiety before monomer polymerization. In some embodiments, the repeating unit of the polymer backbone is attached with the pendant moiety after monomer polymerization. In some embodiments, the monomer is attached with the pendant moiety before chelating the pendant moiety with the element. In some embodiments, the monomer is attached with the pendant moiety after chelating the pendant moiety with the element. In some embodiments, the repeating unit of the polymer backbone is attached with the pendant moiety before chelating the pendant moiety with the element. In some embodiments, the repeating unit of the polymer backbone is attached with the pendant moiety after chelating the pendant moiety with the element. In some embodiments, the method further comprises de-protecting the pendant moiety before chelating with the element.

In some embodiments of the method for preparing the tag of the present disclosure, the end group is attached to an end of the polymer backbone before attaching the pendant moiety to the repeating unit of the polymer backbone. In some embodiment, the end group is attached to an end of the polymer backbone after attaching the pendant moiety to the repeating unit of the polymer backbone. In some embodiments, the end group is attached to an end of the polymer backbone before chelating the pendant moiety with the element. In some embodiments, the end group is attached to an end of the polymer backbone after chelating the pendant moiety with the element. In some embodiments, the end of the polymer backbone is one or more N-terminals of the polymer backbone. In some embodiments, the method further comprises de-protecting the pendant moiety before chelating with the element.

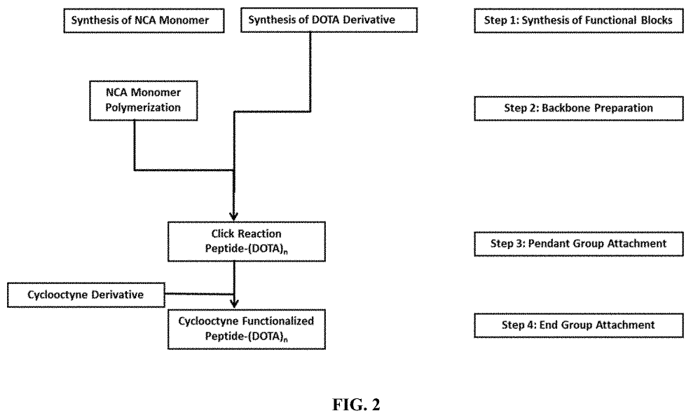

FIG. 2 depicts a non-limiting scheme for synthesizing the tag according to one embodiment of the present disclosure. Step 1 in FIG. 2 describes the synthetic procedure of the functional blocks used in the tag, and said functional blocks include the monomers of the polymer backbone and the pendant moieties. The monomers can be N-Carboxyanhydride derivatives (NCA-derivatives) as described herein, such as azide-NCA, alkynyl-NCA and functional precursor NCA. The pendant moieties can be 1,4,7,10-Tetraazacyclododecane-1,4,7,10-tetraacetic acid derivatives (DOTA-derivatives) as described herein, such as alkynyl-DOTA and azide-DOTA. The DOTA-derivatives may be optionally protected depending upon the specific type of reaction employed, as described herein, such as by tert-butyl. After the pre-functionalization, in step 2, the monomers are polymerized to form a polypeptide backbone. In step 3, DOTA derivatives are attached to the backbone through a click reaction to form polypeptide-(DOTA)n. Depending upon the specific type of click reaction, the polypeptide-(DOTA)n may be optionally deprotected according to the procedures described herein. In step 4, the N-terminal of the polypeptide backbone is attached with an end group such as cyclootyne derivatives.

In some embodiments, the carboxylic groups in DOTA-moiety is protected using tert-butyl or other protecting groups during the CuAAC click reaction procedure to avoid or reduce premature and irreversible sequestering of the catalytic ions.

### Preparation of the Conjugate

In another aspect, the disclosure provides a method for preparing a conjugate for element analysis, comprising (i) pre-functionalizing a biomolecule; and (ii) contacting the tag of the disclosure with the biomolecule.

The biomolecule to be coupled with the tag can be any biomolecule suitable for the analysis of the present disclosure. In particular, the biomolecule for preparing the conjugate can be selected from the group consisting of peptide, protein, aptamer, antibody, enzyme, carbohydrate, nucleic acid, deoxyribonucleic acid, oligonucleotide, polypeptide, recombinant protein, ribonucleic acid lipid, and a derivative thereof.

In some embodiments, the method for preparing the conjugate of the present disclosure comprises pre-functionalizing the biomolecule with a reactive group which can covalently bind to the end group of the tag of the present disclosure.

In some embodiments, the method for preparing the conjugate of the present disclosure comprises pre-functionalizing the biomolecule with an alkyne-reactive group. Examples of the alkyne-reactive groups include but are not limited to azide group and derivatives thereof.

In some embodiments, the method for preparing the conjugate of the disclosure comprises pre-functionalizing the biomolecule with an azide-reactive group such as a cyclooctyne group or a derivative thereof. Examples of the azide-reactive group include but are not limited to DIFO, BCN, DIBAC, DIBO, ADIBO and derivatives thereof.

In some embodiments, the method for preparing the conjugate of the disclosure comprises pre-functionalizing the biomolecule with a tetrazine-reactive group. Examples of the tetrazine-reactive groups include but are not limited to cyclooctene group and derivatives thereof.

In some embodiments, the method for preparing the conjugate of the disclosure comprises pre-functionalizing the biomolecule with a cyclooctene-reactive group. Examples of cyclooctene-reactive groups include but are not limited to tetrazine group and derivatives thereof.

In some embodiments, the biomolecule is coupled with the end group of the tag through a copper-free click reaction.

In some embodiments, the method for preparing the conjugate comprises preparing the tag of the present disclosure. In some embodiments, the method for preparing the conjugate comprises preparing the tag of the present disclosure, pre-functionalizing a biomolecule and coupling the pre-functionalized biomolecule with the tag of the disclosure.

In some embodiments of the method for preparing the conjugate of the present disclosure, the element chelates with the pendant moiety before attaching the pendant moiety to the monomer or the repeating unit of the polymer backbone. In some embodiments, the element chelates with the pendant moiety after attaching the pendant moiety to the monomer or the repeating unit of the polymer backbone. In some embodiments, the element chelates with the pendant moiety of the tag before attaching the end group to the polymer backbone. In some embodiments, the element chelates with the pendant moiety of the tag after attaching the end group to the polymer backbone. In some embodiments, the element chelates with the pendant moiety before coupling the biomolecule with the polymer backbone. In some embodiments, the element chelates with the pendant moiety after coupling the biomolecule with the polymer backbone. In some embodiments, the method further comprises de-protecting the pendant moiety before chelating with the element.

In some embodiments of the method for preparing the conjugate of the present disclosure, the pendant moiety chelates with the element before being attached to the monomer or repeating unit of the polymer backbone. In some embodiments, the pendant moiety chelates with the element after being attached to the monomer or repeating unit of the polymer backbone. In some embodiments, the method further comprises de-protecting the pendant moiety before chelating with the element. In some embodiments, the pendant moiety is attached to the monomer before monomer polymerization. In some embodiments, the pendant moiety is attached to the repeating unit of the polymer backbone after monomer polymerization. In some embodiments, the pendant moiety is attached to the repeating unit of the polymer backbone before attaching the end group to an end of the polymer backbone. In some embodiments, the pendant moiety is attached to the repeating unit of the polymer backbone after attaching the end group to an end of the polymer backbone. In certain embodiments, the end group is attached to one or more N-terminal of the polymer backbone. In some embodiments, the pendant moiety is attached to the repeating unit of the polymer backbone before coupling the biomolecule with the polymer backbone. In some embodiments, the pendant moiety is attached to the repeating unit of the polymer backbone after coupling the biomolecule with the polymer backbone.

In some embodiments of the method for preparing the conjugate of the present disclosure, the monomer is attached with the pendant moiety before monomer polymerization. In some embodiments, the repeating unit of the polymer backbone is attached with the pendant moiety after monomer polymerization. In some embodiments, the monomer is attached with the pendant moiety before chelating the pendant moiety with the element. In some embodiments, the monomer is attached with the pendant moiety after chelating the pendant moiety with the element. In some embodiments, the repeating unit of the polymer backbone is attached with the pendant moiety before chelating the pendant moiety with the element. In some embodiments, the repeating unit of the polymer backbone is attached with the pendant moiety after chelating the pendant moiety with the element. In some embodiments, the polymer backbone is attached with the end group before attaching the pendant moiety to the repeating unit of the polymer backbone. In some embodiments, the polymer backbone is attached with the end group after attaching the pendant moiety to the repeating unit of the polymer backbone. In some embodiments, the polymer backbone is coupled with the biomolecule before attaching the pendant moiety to the repeating unit of the polymer backbone. In some embodiments, the polymer backbone is coupled with the biomolecule after attaching the pendant moiety to the repeating unit of the polymer backbone.

In some embodiments of the method for preparing the conjugate of the present disclosure, the end group is attached to an end of the polymer backbone before attaching the pendant moiety to the repeating unit of the polymer backbone. In some embodiment, the end group is attached to an end of the polymer backbone after attaching the pendant moiety to the repeating unit of the polymer backbone. In some embodiments, the end group is attached to an end of the polymer backbone before chelating the pendant moiety with the element. In some embodiments, the end group is attached to an end of the polymer backbone after chelating the pendant moiety with the element. In some embodiments, the end of the polymer backbone is one or more N-terminals of the polymer backbone. In some embodiments, the end group is coupled with the biomolecule before attaching the pendant moiety to the repeating unit of the polymer backbone. In some embodiments, the end group is coupled with the biomolecule after attaching the pendant moiety to the repeating unit of the polymer backbone. In some embodiments, the end group is coupled with the biomolecule before chelating the pendant moiety with the element. In some embodiments, the end group is coupled with the biomolecule after chelating the pendant moiety with the element. In some embodiments, the method further comprises de-protecting the pendant moiety before chelating with the element.

In some embodiments of the method for preparing the conjugate of the present disclosure, the biomolecule is coupled with the end group of the polymer before attaching the pendant moiety to the repeating unit of the polymer backbone. In some embodiments, the biomolecule is coupled with the end group of the polymer after attaching the pendant moiety to the repeating unit of the polymer backbone. In some embodiments, the biomolecule is coupled with the end group of the polymer before chelating the pendant moiety with the element. In some embodiments, the biomolecule is coupled with the end group of the polymer after chelating the pendant moiety with the element. In some embodiments, the method further comprises de-protecting the pendant moiety before chelating with the element.

In some embodiments, the biomolecule is an antibody, and the pre-functionalization can be performed by incorporating one or more GalNAz (azide-modified galactosamine) groups into one or more glycan chains of the antibody. For example, the pre-functionalization can be performed by incorporating 4 GalNAz groups to the glycan chains of the antibody.

IgG antibodies contain two N-linked glycans attached to specific conserved asparagine residues located in the antibody heavy chain Fc domain. The IgG antibody can be pre-functionalized with azide groups by using chemoenzymatic approach. In particular, β-galactosidase is used to remove terminal Gal residue of glycan chains on antibodies. N-acetylglucosamine (GlcNAc) residues are exposed and ready for activation. GlcNAc residues are functionalized by enzymatic attachment of GalNAz using GalT (Y289L) enzyme.

In some embodiments, the biomolecule including oligonucleotides, synthesized peptide and recombinant protein can be pre-functionalized. In some embodiments, the biomolecule is oligonucleotides, and azide-modified phosphoramidite is used to directly incorporate the azide modifications into the synthetic oligonucleotides. In some embodiments, azide-modified amino acid can be used to incorporate the azide group into a synthesized peptide. In some embodiments, azide groups can be incorporated into a recombinant protein by recombinant, enzymatic, and chemical approaches.

### Kit

In another aspect, the disclosure provides a kit comprising (i) the tag of the disclosure; and (ii) an instruction for using the kit.

As used herein, the term “kit” refers to components packaged or marketed for use together. For example, the kit of the present disclosure can comprise the tag of the disclosure, and an instruction for using the kit in, for example, two containers.

In some embodiments, the kit of the disclosure further comprises an azide reagent for pre-functionalizing a biomolecule and an instruction for performing the pre-functionalization. In some embodiments, the kit may further comprises an azide reagent for pre-functionalizing a peptide, protein, aptamer, antibody, enzyme, carbohydrate, nucleic acid, deoxyribonucleic acid, oligonucleotide, polypeptide, recombinant protein, ribonucleic acid lipid, and/or a derivative thereof, and an instruction for performing the pre-functionalization for the peptide, protein, aptamer, antibody, enzyme, carbohydrate, nucleic acid, deoxyribonucleic acid, oligonucleotide, polypeptide, recombinant protein, ribonucleic acid lipid, and/or a derivative thereof.

In some embodiments, the kit of the disclosure comprises a biomolecule. In some embodiments, the kit of the disclosure comprises a peptide, protein, aptamer, antibody, enzyme, carbohydrate, nucleic acid, deoxyribonucleic acid, oligonucleotide, polypeptide, recombinant protein, ribonucleic acid lipid, and/or a derivative thereof, and an instruction for performing the pre-functionalization for the peptide, protein, aptamer, antibody, enzyme, carbohydrate, nucleic acid, deoxyribonucleic acid, oligonucleotide, polypeptide, recombinant protein, ribonucleic acid lipid, and/or a derivative thereof to couple with the tag of the kit.

In some embodiments, the kit may further comprise a catalyst for chelating the tag with the element. For example, the kit may further comprise a metal solution, such as a copper solution to catalyze the chelation of the tag with an element.

In some embodiments, the metal solution is a copper solution comprising copper nitrate, copper formate, copper nitrite, copper nitride, copper cyanide, copper ferrocyanide, copper chloride, copper bromide, copper perchlorate, copper bromate, copper iodide, copper sulfide, copper sulfate, copper thiocyanate, copper carbonate, copper acetate, copper oxalate, copper butyrate, copper citrate, copper benzoate, copper borate, copper phosphate, copper carbide, copper chromate, copper tungstate, and any mixture thereof.

### EXAMPLE

The disclosure is further illustrated by the following examples. The examples are provided for descriptive purposes only, and are not to be construed as limiting the scope or content of the disclosure in any way.

### Example 1 Synthesis of the Functional Blocks

### 1.1. Synthesis of NCA-Derivatives (Azide-NCA, Alkynyl-NCA and Functional Precursor NCA)

The NCA monomers used in backbone preparation may include two categories of pre-functionalized NCA monomers (azide-NCA, alkynyl-NCA) and functional precursor NCA. The synthetic procedures of each NCA monomers are illustrated below, separately.

### 1.1.1. Synthesis of Azide-NCA

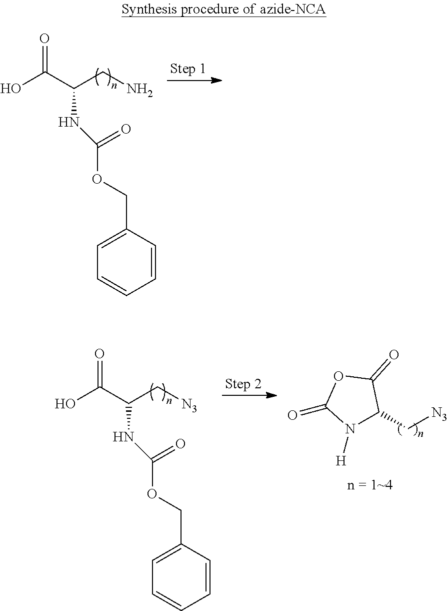

### Step 1: Synthesis of N-α-carboxybenzyl-L-azidonorleucine

K₂CO₃ (2.66 g, 19.3 mmol) was dissolved in THF/H₂O (1:1, 80 mL). CuSO₄.5H₂O (18 mg, 0.072 mmol) was added to the stirring solution followed by the addition of N-α-carboxybenzyl-L-Lysine (6.48 g, 24.2 mmol). Imidazole-1-sulfonyl-azide-HCl (1.78 g, 8.57 mmol) was added and the reaction mixture was stirred at room temperature for a period of 12h. THF was removed under reduced pressure and the reaction mixture was neutralized with 10% HCl to a pH of 5. The aqueous layer was extracted with EtOAc (3×50 mL), organic layers combined, dried with anhydrous MgSO₄, filtered, and concentrated. The product was isolated as a clear oil. No further purification was required. ¹H-NMR (500 MHz, CDCl₃): δ=7.41-7.37 (m. 5H), 5.37 (s, 1H), 5.13 (m, 2H), 4.44 (m, 2H), 3.29-3.27 (m, 2H), 1.91-1.76 (m, 2H), 1.76-1.65 (m, 2H), 1.63-1.61 (m, 2H).

### Step 2: Synthesis of L-Azidonorleucine NCA

N-α-carboxybenzyl-L-azidonorleucine (650 mg, 2.12 mmol) was added to a 125 mL Schlenk flask equipped with a Teflon stir bar and then dissolved in 50 mL THF. Ghosez's reagent (439 μL, 3.32 mmoL) was added via syringe to the stirring solution under N₂. The reaction was stirred at room temperature until completion (48h). The solution was removed under reduced pressure and dissolved in 10 mL cold EtOAc and extracted with cold 5% sodium bicarbonate solution (3×10 mL). The organic layer was separated, diluted in cold EtOAc (10 mL) and dried with anhydrous MgSO₄. The solution was removed under reduced pressure and transferred to a glovebox (N₂ atmosphere) for further purification. The residue was purified by flash chromatography (10% to 75% THF in hexanes) and collected in 10×10 mL fractions. 280 mg of NCA was isolated as a clear oil after evaporation of the THF and hexanes from the combined fractions. ¹H-NMR (500 MHz, CDCl₃): δ=6.40 (s, 1H), 4.37-4.34 (t, J=5.5, 1H), 3.40-3.33 (t, J=2.0, 2H), 1.99-1.81 (m, 2H), 1.67-1.58 (m, 2H), 1.55-1.49 (m, 1H).

### 1.1.2. Synthesis of Alkynyl-NCA

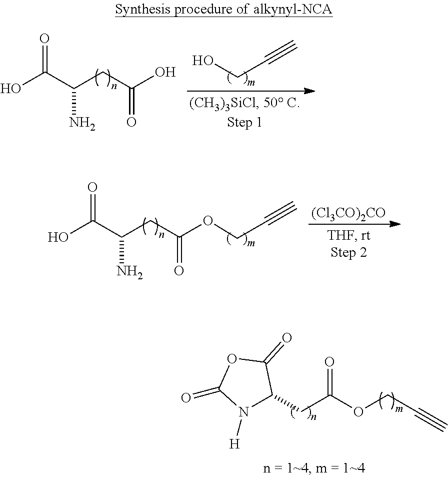

### Step 1: Synthesis of γ-Propargyl L-Glutamate Hydrochloride

L-glutamic acid (10 g, 68 mmol) was suspended in propargyl alcohol (330 mL) under Ar. Chlorotrimethylsilane (17.27 mL, 136 mmol) was added dropwise to the suspension. The resulting suspension was heated to 50° C. and stirred until it became homogeneous. The solvent was removed at 60° C. under vacuum. The reaction solution was precipitated into diethyl ether giving a white solid. ¹H-NMR (400 MHz, D2O) δ=2.20 (m, 2H, CH2), 2.63 (dt, 2H, CH—CO), 2.86 (t, 1H, C≡CH), 4.05 (t, 1H, CH), 4.69 (d, 2H, CH₂CO).

### Step 2: Synthesis of NCA of γ-Propargyl-L-Glutamate

γ-propargyl-L-glutamate hydrochloride (1.35 g, 6.13 mmol) was suspended in dry ethyl acetate (50 mL) and the solution was heated to reflux. Triphosegene (0.61 g, 2.04 mmol) was added and the reaction was refluxed for 4-5 hours under N₂. The reaction solution was cooled to room temperature and any unreacted γ-propargyl-L-glutamate hydrochloride was removed by filtration. The reaction solution was then cooled to 5° C. and washed with 50 mL of water, 50 mL of saturated sodium bicarbonate, and 50 mL of brine all at 5° C. The solution was then dried with anhydrous magnesium sulfate, filtered, and concentrated down to viscous oil. ¹H-NMR (400 MHz, CDCl₃) δ=2.20 (dm, 2H, CH2), 2.49 (t, 1H, C≡CH), 2.58 (t, 2H, CHCO), 4.39 (t, 1H, CH), 4.68 (d, 2H, CH₂CO), 6.5 (s, 1H, NH).

### 1.1.3. Synthesis of Functional Precursor NCA

The functional precursor NCA is a monomer that can be further functionalized using azide or alkynyl after polymerization and clickable after further functionalization.

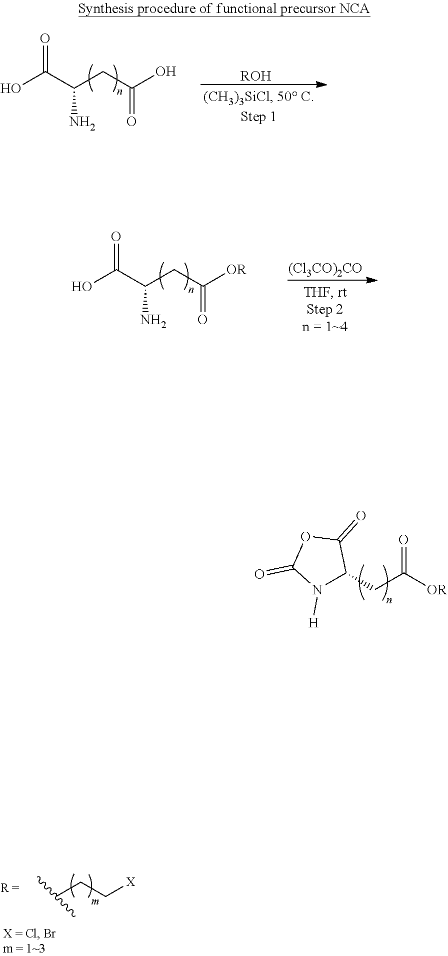

### Step 1: Synthesis of γ-Chloropropanyl-L-Glutamate

L-Glutamic acid (11.04 g, 75 mmol) and 3-chloropropanol (80 mL, 0.96 mol) were mixed in a round-bottom flask (250 mL), followed by addition of chlorotrimethylsilane (10.5 mL, 83 mmol) via syringe. The resulting suspension was heated to 50° C. and stirred until it became homogeneous. The solvent was removed at 60° C. under vacuum. Addition of diethyl ether (40 mL) to the residue yielded a white solid which was collected by filtration. Additional purification by recrystallization in ethanol/diethyl ether afforded the final product as a white solid.

### Step 2: γ-3-Chloropropanyl-L-Glutamic NCA

A round-bottomed flask (25 mL) was charged with γ-chloropropanyl-L-glutamate 2 (1.0 g, 4.5 mmol), triphosgene (0.8 g, 2.7 mmol), and anhydrous THF (15 mL) under nitrogen. The mixture was stirred at room temperature for 24h over which period the γ-chloropropanyl-L-glutamate was gradually dissolved. Removal the solvent under vacuum yielded an oily liquid which was then dissolved in ethyl acetate (20 mL) and washed with a cold saturated NaHCO₃/H₂O solution. The organic layer was separated and dried over anhydrous MgSO₄ at 0° C. Filtration, evaporation, and recrystallization in CH₂Cl₂/hexane at −85° C. in a deep freezer afforded the final product as a white solid.

### 1.2. Synthesis of Functionalized DOTA-Derivatives (Alkynyl-DOTA-(tBu) and Azide-DOTA-(tBu))

### 1.2.1. Synthesis of Alkynyl-DOTA-(tBu)

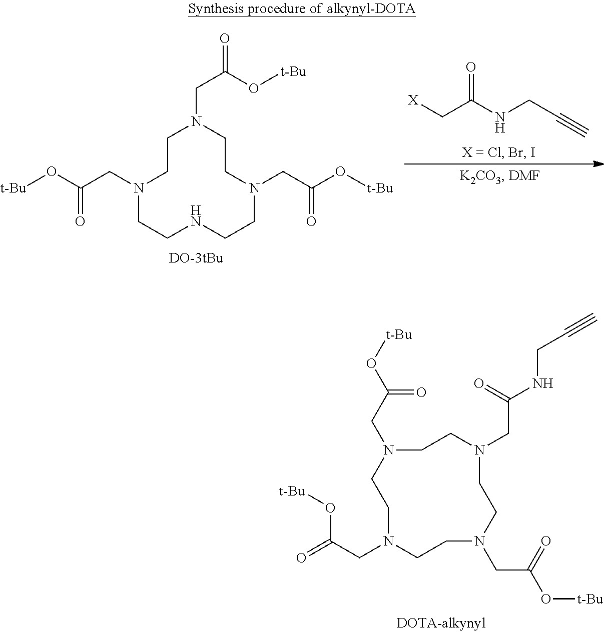

A solution of DO-3tBu (0.100 g, 0.194 mmol), N-(2-propynyl)-chloroacetamide (0.026 g, 0.194 mmol) and K₂CO₃ (0.028 g, 0.194 mmol) in DMF (2 ml) was stirred under nitrogen at room temperature for 2 h. After removal of the solvent in vacuum, the residual mixture was purified by silica gel column using chloroform-methanol (98:2) to give a colorless solid (0.092 g, 78%).

DO-3tBu (1.48 g, 2.48 mmol), N-(2-propynyl)-bromoacetamide (1.29 g, 7.33 mmol), and

K₂CO₃ (2.78 g, 20.1 mmol) were placed in a 500 mL flask and the mixture was then dissolved in CH₃CN (HPLC grade, 150 mL). The reaction mixture was heated at reflux for 48 hours, cooled to room temperature, and the solvent was removed by rotary evaporation. CH₂Cl₂ (50 mL) was added, the mixture was filtered through filter paper, and then evaporated to dryness, yielding crude DOTA-alkynyl as a golden oil. The residual mixture was purified by silica gel column using chloroform-methanol (98:2) to give a colorless solid.

### 1.2.2. Synthesis of Azide-DOTA-(tBu)

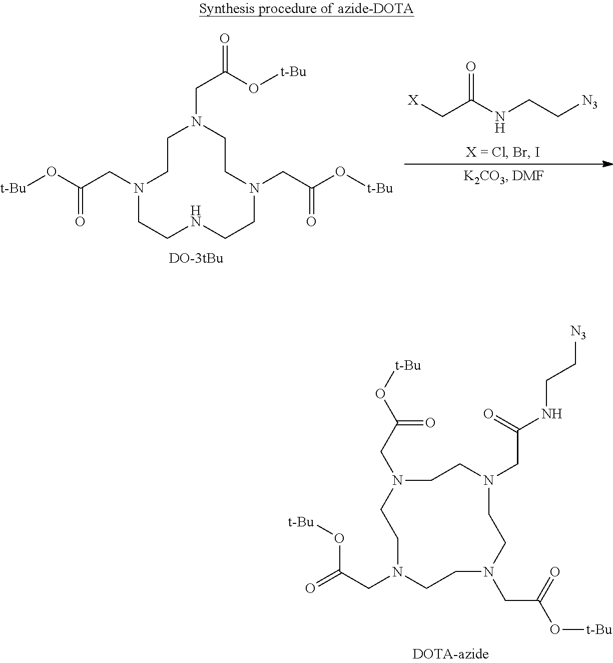

DO-3 tBu (257 mg, 0.5 mmol) and N-chloroacetyl-2-azidoethylamine (89 mg, 0.55 mmol) were dissolved in MeCN (4 ml), followed by the addition of K₂CO₃ (180 mg, 1.3 mmol). The mixture was stirred for 18h at 60° C. The solvent was evaporated and the residue was partitioned between water (20 ml) and EtOAc (2×20 ml). Combined organic extract was dried and was concentrated, the residue was triturated with hexanes to afford the azide modified ligand azide-DOTA-(tBu). ¹H-NMR (CDCl₃) δ=9.24 (s, D₂O exch., 1H), 3.42-2.23 (m, 28H), 1.45 (m, 27H).

### Example 2 Backbone Preparation

The peptide-based backbone was prepared via NCA polymerization.

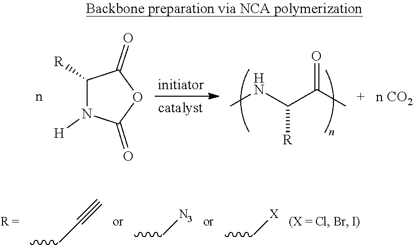

2.1. Synthesis of Alkynyl-Polypeptide from Alkynyl-NCA

To a solution of alkynyl-NCA (γ-propargyl-L-glutamate NCA, 800 mg, 4.37 mmol) and anhydrous LiBr (190 mg, 2.2 mmol) were dissolved in 28 ml dry DMF in a Schlenk tube. A solution of benzylamine (2.2 mg, 20 μmol) in 2 ml of dry DMF was added after NCA and LiBr were totally dissolved. The reaction was maintained for 5 days at 0° C. under an inert atmosphere. The reaction mixture was precipitated into an excess diethyl ether, filtered and dried under vacuum to yield a pale yellow solid, PDI=1.36.

Alternatively, in a dry box, alkynyl-NCA (γ-propargyl-L-glutamate NCA, 20.7 mg, 0.1 mmol) was dissolved in dry DMF (0.5 ml). The alkynyl-NCA solution was then added to a DMF solution containing N-TMS benzylamine (10 μL, 0.1 mmol/mL). The reaction mixture was stirred for 24 h at room temperature. After alkynyl-NCA was completely consumed (monitored by checking the NCA anhydride band at 1790 cm⁻¹ using FT-IR), the reaction mixture was precipitated into an excess diethyl ether, filtered and dried under vacuum to yield a pale yellow solid, PDI=1.21.

Alternatively, in a dry box, alkynyl-NCA (γ-propargyl-L-glutamate NCA, 20.7 mg, 0.1 mmol) was dissolved in dry DMF (0.5 ml). The alkynyl-NCA solution was then added to a DMF solution containing hexamethyldisilazane (HMDS) (10 μL, 0.1 mmol/mL). The reaction mixture was stirred at room temperature for 24h. After quenching the reaction by exposure to air, the solution was removed at 40° C. under vacuum. The product was further purified by dissolution in CH₂Cl₂ and precipitation from methanol. Filtration and drying under vacuum at 40° C. for 48h yielding the final product as a white solid, with PDI=1.21.

TABLE 1
HMDS mediated ROP of γ-propargyl-L-glutamate NCA in DMF^{a)}

| | | Mn (×10³ g/mol) | | | Conversion |
|---|---|---|---|---|---|
| No | [M]₀/[I]₀ | Calc ^{b)} | GPC ^{c)} | PDI ^{c)} | (%) ^{d)} |
| 1 | 25 | 4.2 | 4.4 | 1.24 | 99 |
| 2 | 50 | 8.2 | 7.9 | 1.26 | 97 |
| 3 | 100 | 16.0 | 14.9 | 1.21 | 95 |
| 4 | 200 | 30.2 | 28.7 | 1.17 | 90 |

^{a)}Polymerization ([M]₀ = 0.2M) were conducted in DMF at room temperature for 24 h using HMDS as initiator.
^{b)} The calculated molecular weight was calculated according to the ratio of [M]₀/[I]₀ and the conversion.
^{c)} The absolute molecular weight and PDI of polymer was determined from GPC using DMF as solvent.
^{d)} The conversion was determined from ¹H-NMR spectroscopy.

2.2. Synthesis of Azide-Polypeptide from Azide-NCA W2.40 The polymerization reactions were performed in a dinitrogen filled glove box. To a solution of azide-NCA (azidenorleucine-NCA) (20 mg, 0.10 μmol) in dry THF (500 μL) was rapidly added via syringe, a solution of (PMe₃)₄Co in dry THF (120 μL, 6.8 μmol). The reaction was stirred at room temperature. Polymerization reactions were generally completed within 1 hour. Reactions were removed from the dry-box, all THF was removed, and the polypeptide was washed with 100 mM HCl (2×15 mL), centrifuged for 5 minutes at 3000 rpm and the supernatant was removed. The white solid polypeptide was washed with 10 mL water and then lyophilized to yield azide-polypeptide as a white solid. ¹H-NMR (500 MHz, TFA-d): δ=5.87 (s, 1H), 5.27 (s, 2H), 4.60 (s, 3H), 3.36 (s, 2H), 2.70 (s, 2H), 2.16 (s, 3H), 1.87-1.63 (s, 2H), 1.62-1.48 (s, 3H).
2.3. Synthesis of Azide-Polypeptide from Functional Precursor NCA

### Step 1: Polymerization of γ-3-Chloropropanyl-L-Glutamic NCA

In a glovebox, γ-3-chloropropanyl-L-glutamic NCA (107 mg, 0.429 mmol) was dissolved in DMF (1.4 mL). A measured volume of HMDS/DMF stock solution (C₁=57.4 mM, 75 μL, 4.29 μmol) was subsequently added with syringe. The reaction mixture was stirred at room temperature for 24 h and quenched by exposure to air. DMF was removed at 40° C. under vacuum to yield a polymer film. The polymer film was further purified by dissolution in CH₂Cl₂ (2 mL) and precipitation from methanol (20 mL). Filtration and drying under vacuum at 60° C. for 8h afforded the final product as a white solid. ¹H-NMR (CDCl₃-d) δ=4.22 (br s, 3H, ClCH₂CH₂CH₂— and CHNH), 3.61 (s, 2H, ClCH₂CH₂CH₂—), 2.67 (brs, 2H, —COCH₂CH₂—), 2.38 (br s, 2H, —COCH₂CH₂—), 2.09 (s, 2H, ClCH₂CH₂CH₂—).

### Step 2: Synthesis of Poly(γ-3-Azidopropanyl-L-Glutamate)

A DMF (5 mL) solution of poly(γ-3-chloropropanyl-L-glutamate) (0.1 g, 0.49 mmol) and sodium azide (0.3 g, 4.6 mmol) was stirred at 60° C. for two days and allowed to cool to room temperature. The reaction mixture was passed through a neutral alumina column to remove any inorganic salts. DMF was removed by vacuum distillation at 60° C. to yield a polymer film. The polymer film was further purified by dissolution in CH₂Cl₂ and precipitation in methanol. The resulting polymer was collected by filtration and dried at 60° C. under vacuum. ¹H-NMR (CDCl₃-d): δ=4.18 (s, 2H, N₃CH₂CH₂CH₂—), 3.95 (br s, 1H, CHNH), 3.40 (s, 2H, N₃CH₂CH₂CH₂—), 2.68 (br s, 2H, —COCH₂CH₂—), 2.39 (br s, 2H, —COCH₂CH₂—), 1.92 (s, 2H, N₃CH₂CH₂CH₂—).

### Example 3 Pendent Group Modification

The DOTA decorated polypeptide-(C—N)-DOTA-(tBu) and poly-peptide-(N—C)-DOTA-(tBu) were synthesized from alkynyl-polypeptide and azide-polypeptide separately via CuAAC click reaction as the combination shown in Table 2 below to form polypeptide-DOTA-(tBu). In this example, the carboxylic groups in DOTA-moiety was protected using tert-butyl or other protecting groups during the CuAAC click reaction procedure to avoid premature and irreversible sequestering of the catalytic ions.

TABLE 2
Reactant combination

| | alkynyl-Polypeptide | azide-Polypeptide |
|---|---|---|
| alkynyl- | | Polypeptide- |
| DOTA-(tBu) | | (N—C)-DOTA-(tBu) |
| alkynyl- | Polypeptide-(C—N)-DOTA-(tBu) | |
| DOTA-(tBu) | | |

In a glovebox poly(γ-propargyl-L-glutamate) (18.3 mg) and azide-DOTA-(tBu) (128 mg, 0.2 mmol) were dissolved in DMF (5 ml). Then 200 μL PMDETA (10 μmol, 50 mM DMF solution) and 200 μL Cu(I)Br (10 μmol, 50 mM DMF solution) were subsequently added into the stirring solution. The reactant molar ratio was alkyne/azide/CuBr/PMDETA 1:2:0.1:0.1. The reaction mixture was stirred at room temperature for 24 h and quenched by exposure to air. After the reaction was completed, the reaction solution was passed through a short aluminum oxide column to remove the catalyst. The functionalized polypeptide was purified by dialysis and lyophilized to solid.

### Example 4 Deprotection

The mixture TFA/TIPS/H2O 95:2.5:2.5 was used as deprotection reagent to remove the t-Bu group on polypeptide-DOTA-(tBu).

Polypeptide-DOTA-(tBu) was stirred in TFA/TIPS/H₂O 95:2.5:2.5 for 3h at room temperature. The product was precipitated with ice cold ether and purified by semipreparative high performance liquid chromatography.

### Example 5 End Group Modification

The final product was synthesized by decorating the terminal amino group using cyclooctyne-derivatives via amidation reaction.

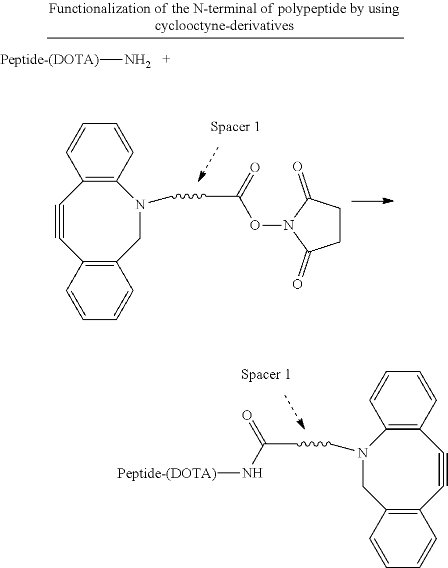

In a glovebox, polypeptide-DOTA (50 mg) and isopropylethylamine (200 μl) were dissolved in DMF (2 ml). 200 μl dibenzocyclooctyne-N-hydroxysuccinimidyl ester (10 μmol, 50 mM DMF solution) was subsequently added to the solution. The product was precipitated with ice cold ether and purified by semipreparative high performance liquid chromatography.

### Example 6 Preparation of a Metal Tag

The metal used in the element tag can be any element capable of chelating with DOTA. Lanthanides are preferred due to their low abundancy in environment.

Briefly, polymer (200m) was dissolved in water (95 μL) and mixed with 50 mM metal solutions (5 μL). The mixed solution was stirred at room temperature for 1 hour. Free metal ions were washed away by using Amicon Ultra centrifugal filter (3 kD cutoff).

### Example 7 Pre-Functionalization of the Biomolecule with Azide

Antibody: IgG antibodies contain two N-linked glycans attached to specific conserved asparagine residues located in the antibody heavy chain Fc domain. The IgG antibody can be pre-functionalized with azide by using chemoenzymatic approach.

Briefly, β-galactosidase was used to remove terminal Gal residue of glycan chains on antibodies. N-acetylglucosamine (GlcNAc) residues were exposed and ready for activation. GlcNAc residues was functionalized by enzymatic attachment of GalNAz (azide-modified galactosamine) using GalT(Y289L) enzyme. On average, each antibody had two heavy chains, and 4 GalNAz residues were attached.

In addition to antibodies, other pre-functionalized biomolecule, including oligonucleotides, synthesized peptides and recombinant proteins can also be used. For example, azide-modified phosphoramidite was used and resulted in direct incorporation of azide modifications into synthetic oligonucleotides, and azide groups were also successfully used to modify recombinant proteins via recombinant, enzymatic, and chemical approaches.

### Example 8 Conjugation of the Metal Labeled Polypeptide with the Azide Pre-Functionalized Biomolecule Via Copper-Free Click Reaction

The peptide-based metal tag was stoichiometrically connected with the biomolecule via copper-free click reaction, as shown below.

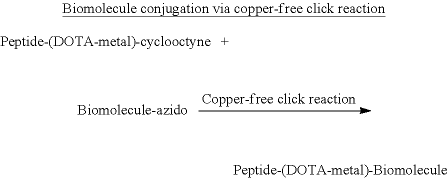

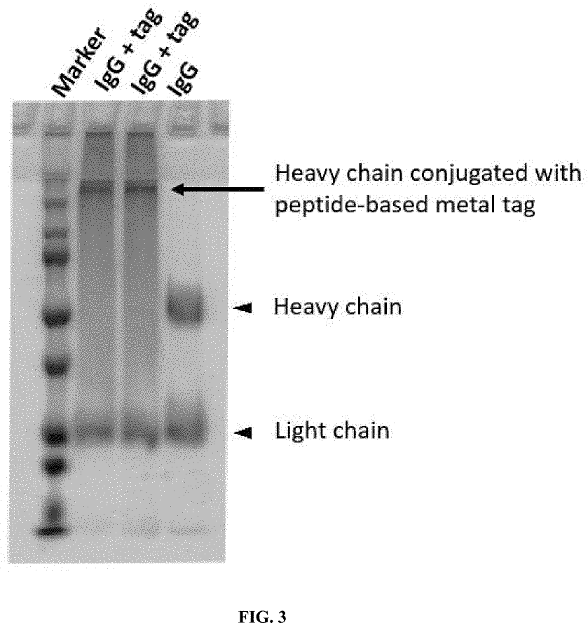

Briefly, pre-functionalized IgG prepared in Example 7 was subject to copper-free click reaction with the peptide-based metal tag as described above. After the reaction, the product and IgG molecule without conjugation were both loaded on SDS-PAGE gel to determine whether the IgG was conjugated with the peptide-based metal tag. As can be seen from FIG. 3, the peptide-based metal tag was successfully conjugated with the heavy chains of the IgG, and the conjugates are the bands annotated with the arrow.

### Example 9 Quantification of Cell Surface Markers by Using Antibodies Conjugated with the Element Tags of the Present Disclosure

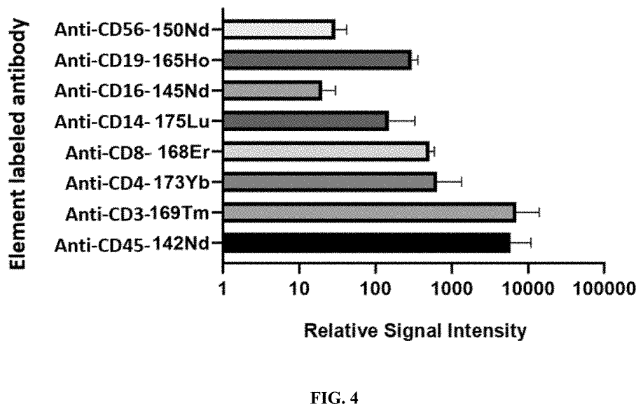

Anti-CD56 antibody, anti-CD19 antibody, anti-CD16 antibody, anti-CD14 antibody, anti-CD8 antibody, anti-CD4 antibody, anti-CD3 antibody and anti-CD45 antibody were conjugated with element tags chelating with 150Nd, 165Ho, 145Nd, 175Lu, 168Er, 173Yb, 169Tm, and 142Nd according to the method of Example 8, respectively. The resulted conjugates were incubated with human peripheral blood mononuclear cells expressing CD56, CD19, CD16, CD14, CD8, CD4, CD3 and/or CD45 on the surface. After incubation, the cells were collected and subject to elemental analysis. Quantification of each cell surface marker was performed based on the relative signal intensity of each element of the corresponding element tag. The result is as shown in FIG. 4.

While this disclosure has been described with an emphasis on preferred embodiments, it will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with the true scope and spirit of the disclosure being defined by the following claims.

1.-125. (canceled)

126. A tag for labeling a biomolecule comprising a polymer backbone terminating into an end group capable of stoichiometrically binding to the biomolecule, and one or more pendant moieties each attached to the polymer backbone and capable of chelating with an element.

127. The tag of claim 126, wherein the polymer backbone is selected from the group consisting of polypeptide, polypeptoid, poly β-peptide, poly γ-peptide, poly δ-peptide, and a derivative thereof.

128. The tag of claim 126, wherein the polymer backbone is a homopolymer or a copolymer.

129. The tag of claim 126, wherein the polymer backbone is formed by polymerization of a monomer selected from the group consisting of NCA, NTAα-amino acid, β-amino acid, γ-amino acid, δ-amino acid, N-substituted amino acid, and a derivative thereof.

130. The tag of claim 126, wherein the pendant moiety is selected from the group consisting of EDTA, DTPA, DCTA, DOTA, TETA, NOTA and a derivative thereof.

131. A tag for labeling a biomolecule comprising a polymer backbone terminating into an end group capable of binding to the biomolecule, and one or more pendant moieties each attached to the polymer backbone and capable of chelating with an element, wherein the tag is bio-compatible.

132. The tag of claim 131, wherein the polymer backbone is selected from the group consisting of polypeptide, polypeptoid, poly β-peptide, poly γ-peptide, poly δ-peptide, and a derivative thereof.

133. The tag of claim 131, wherein the polymer backbone is formed by polymerization of a monomer selected from the group consisting of NCA, NTA, α-amino acid, β-amino acid, γ-amino acid, δ-amino acid, N-substituted amino acid, and a derivative thereof.

134. The tag of claim 131, wherein the polymer backbone has a polydispersity index of less than 1.4.

135. The tag of claim 131, wherein the number of the pendant moieties attached to the polymer backbone is between 10 and 1000.

136. The tag of claim 131, wherein the pendant moiety is selected from the group consisting of EDTA, DTPA, DCTA, DOTA, TETA, NOTA, and a derivative thereof.

137. The tag of claim 131, wherein each of the one or more pendant moieties is directly attached to the polymer backbone.

138. The tag of claim 131, wherein each of the one or more pendant moieties is attached to the polymer backbone through a linker.

139. The tag of claim 131, wherein the linker is attached to the polymer backbone or the pendant moiety via a spacer.

140. A tag for labeling a biomolecule comprising a polymer backbone terminating into an end group capable of binding to the biomolecule, and one or more pendant moieties each stoichiometrically attached to a repeating unit of the polymer backbone and capable of chelating with an element.

141. The tag of claim 140, wherein the pendant moiety is capable of chelating with a metal or an isotope thereof.

142. The tag of claim 140, wherein the number of the pendant moieties attached to the polymer backbone is between 10 and 1000.

143. The tag of claim 140, wherein the pendant moiety is selected from the group consisting of EDTA, DTPA, DCTA, DOTA, TETA, NOTA, and a derivative thereof.

144. The tag of claim 140, wherein each of the one or more pendant moieties is directly attached to the polymer backbone.

145. The tag of claim 140, wherein each of the one or more pendant moieties is attached to the polymer backbone through a linker.
